(19) United States
(12) Patent Application Publication
(10) Pub. No.: US 2026/0296993 A1
(43) Pub. Date: Oct. 1, 2026

(54) CONTINUOUS DEOXYFLUORINATION WITH SF4

(71) Applicant: LONZA LTD, Visp (CH)

(72) Inventors: Paul HANSELMANN, Basel (CH); Ryan LITTICH, Basel (CH); Michael BERSIER, Basel (CH); Dominique ROBERGE, Basel (CH); Dominik POLTERAUER, Basel (CH); Christopher HONE, Basel (CH); Oliver KAPPE, Basel (CH)

(21) Appl. No.: 19/480,165

(22) PCT Filed: May 1, 2024
(86) PCT No.: PCT/EP2024/061989

§ 371 Date: Oct. 30, 2025

(30) Foreign Application Priority Data
May 3, 2023 (EP) 23171403.1

(51) Int. Cl. C07C 17/16; C07B 39/00; C07C 17/18; C07C 51/60

(52) U.S. Cl. CPC C07C 17/16; C07B 39/00; C07C 17/18; C07C 51/60

(57) ABSTRACT

The present invention relates to a method for continuous deoxyfluorination of certain alcohols, aldehydes, ketones and carboxylic acids with the fluorination reagent SF₄ in the presence of a base in a continuous flow reaction mode.

### FIELD OF THE INVENTION

The present invention relates to a method for continuous deoxyfluorination of certain alcohols, aldehydes, ketones and carboxylic acids with the fluorination reagent SF₄ in the presence of a base in a continuous flow reaction mode.

### BACKGROUND OF THE INVENTION

Fluorinated organic compounds are used as pharmaceutically active substances and as agrochemicals.

W. C. Smith in Ang. Chem. Int. Ed., 1962, 1, 467-518 gives an overview of SF₄ and its use. W. C. Smith et al. in J. Am. Chem. Soc. 1959, 81, 3165-3166 (Smith) and thereafter W. R. Hasek, et al. in J. Am. Chem. Soc. 1960, 82, 543-551 (Hasek) disclose the deoxyfluorination of organic carbonyl and hydroxyl compounds, wherein one hydroxyl is substituted by one fluorine or one oxygen by two fluorines. Hasek discloses in the first column of the article general reaction schemes: for example that the hydroxyl group of carboxylic acids is converted in a first reaction with a first molar equivalent SF₄ to the acyl fluoride group and then further in a second reaction the carbonyl oxygen is converted with one further molar equivalent of SF₄ on to a trifluoromethyl group. Aldehydes and ketones are converted with one molar equivalent SF₄ to the respective geminal difluoro compounds. Alcohols are converted with one molar equivalent SF₄ to the respective fluorides. Hasek reports in table V the conversion of benzaldehyde in the presence of two molar equivalents of SF₄ to benzal fluoride at 150° C. for 6 h with a yield of 81%. Aliphatic aldehydes which possess alpha-hydrogen are reported in table V to give lower yields, e.g. acetaldehyde reacts at 50° C. after 14 h with a yield of 35%. The reactions disclosed in Hasek were done without addition of a base, instead HF was sometimes used as catalyst. A solvent was only used exceptionally. Hasek gives for two alcohols in Table IX yields of 28% and 57% respectively, and states for aliphatic alcohols: “Compounds even less acidic, such as methyl, ethyl and isopropyl alcohols, gave the corresponding alkylfluorides, but in these cases a major byproduct was the alkyl ether” so there are drawbacks in the methods disclosed by Hasek.

T. Gustafsson et al. in Chem. Commun., 2008, 3022-3024 (Gustafsson), discloses a continuous deoxyfluorination method with diethylaminosulfur trifluoride (DAST) as fluorinating agent which is said to be probably the most widely used commercially available nucleophilic fluorinating reagent. As acknowledged in Gustafsson, DAST bears a high risk of explosion when handled at elevated temperatures, which was the motivation in Gustafsson to develop a continuous microreactor-based process for DAST as fluorinating agent, to mitigate the explosion risk. DAST is liquid at room temperature (boiling point 30 to 32° C.) which makes handling less complicated compared to fluorination reagents which are gaseous at room temperature, such as SF₄ (boiling point −38° C.). DAST is prepared from SF₄, so by using SF₄ as fluorinating agent instead of DAST the respective additional preparation step of required for DAST can be omitted.

With respect to the stereoselectivity of the deoxyfluorination reaction with DAST, Gustafsson reports a 6:1 mixture of diastereoisomers in the case of menthol and a 5:1 mixture of diastereoisomers in the case of dehydrocholesterol. Disclosure of diastereomeric mixtures of products, from reactions of diastereomerically pure substrates, demonstrates a non-stereospecific reaction. Chiral separations may therefore be required, following application of the DAST method of Gustafsson.

M. Bugera et al. in J Org. Chem. 2019 84 16105-16115 discloses deoxofluorination with SF₄ of Aliphatic Carboxylic Acids; the carboxylic acid residue is converted to a trifluoromethyl residue.

S. Trofymchuk et al. in J. Org. Chem. 2020, 85, 3110-3124 discloses deoxofluorination with SF₄ of (Hetero)aromatic Acids; the carboxylic acid residue is converted to a trifluoromethyl residue.

S. Trofymchuk et al. in J. Org. Chem. 2021, 86, 12181-12198 discloses a deoxofluorination of substituted acetophenones with sulfur tetrafluoride (SF₄) providing the respective geminal difluoro compounds.

The disclosed reactions are conducted under acidic conditions; HF is set free in stoichiometric amounts and in some instances HF is added as solvent or as catalyst. Reactions under acidic conditions have a scope that is limited to substrates and products which are stable under acidic conditions.

A. F. Janzen et al. in Journal of Fluorine Chemistry, 1988, 38, 205-208 (Janzen) discloses a batch reaction at −50° C. of SF₄ with
(2-furyl)methanol in dichloromethane in the presence of triethylamine with a yield of 18%; and
2-phenylethanol in cyclohexane in the presence of pyridine with a yield of 35%.

The reactions were done in a sealed glass tube at −50° C. providing after 5 min a homogeneous brown solution. The sealing and any residual SF₄ were removed and the product mixture distilled at atmospheric pressure at 55-90° C. giving a black decomposed product. Y. Tamboli et al. in Org. Proc. Res. Dev. 2020, 24, 1609-1613 (Tamboli) discloses nosyl fluoride (13) as a deoxyfluorinating agent for N-Boc-4-hydroxy-L-proline methyl ester (2), reported yield is 52%.

There is a need for a method that is not limited to substrates or products that are stable under acidic conditions. Furthermore there is a need for a method that provides good results with respect to conversion, yields, selectivity and stereoselectivity and which allows for short reaction times.

The inventors of present invention found a method for continuous deoxyfluorination of alcohols, aldehydes, ketones and carboxylic acids as substrates, wherein the substrates and/or obtained fluorinated products can also be unstable under acidic conditions. The method of the present invention provides good results such as conversion, yields, selectivity and stereoselectivity by employing SF₄ as fluorinating agent in the presence of a base. Also aliphatic alcohols can be converted with high yield and selectivity. In addition the method provides for shorter residence times. The risk associated with the handling of DAST is eliminated completely. A step of manufacturing DAST from SF₄ is omitted.

### SUMMARY OF THE INVENTION

Subject of the invention is a method for deoxyfluorination of anyone of substrates a), b), c) and d),
by a reaction of said substrate a), b), c) or d) with the fluorination reagent SF₄ in the presence of at least 1 equiv of a base, the equiv are molar equiv based on the molar amount of substrate;
the reaction is done in a continuous flow reaction mode;
wherein
   substrate a) is a primary or secondary alcohol, or substrate a) is a di-, tri- or tetra-peptide of natural alpha amino acids containing a primary or secondary aliphatic OH residue in its side chain,
   substrate b) is an aldehyde,
   substrate c) is a carboxylic acid, and
   substrate d) is an unsubstituted or substituted cyclohexanone of formula (D6) or an unsubstituted or substituted cyclohexenone of formula (D6), or an unsubstituted or substituted cyclopentanone of formula (D5) or an unsubstituted or substituted cyclopentenone of formula (D5),
and wherein
in case of substrate a) the hydroxyl residue of the primary or secondary alcohol is exchanged against fluorine providing the respective (mono)fluorine compound;
in case of substrate b) the oxo residue of the aldehyde group of the aldehyde is exchanged against two geminal fluorine atoms providing the respective difluoro compound;
in case of substrate c) the hydroxyl residue of the carboxylic acid is exchanged against fluorine providing the respective acid fluoride;
in case of substrate d) the oxo residue of the keto group is exchanged against fluorine providing a vinyl fluoride compound of formula (D6-F) or of formula (D5-F) respectively according to Scheme D,

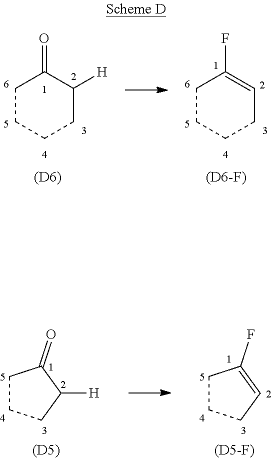

wherein in formula (D6) and formula (D6-F) the three endocyclic bonds C³-C⁴, C⁴-C⁵ and C⁵-C⁶ are either single bonds or one of said three bonds is a double bond and the other two bonds of said three bonds are single bonds, and
wherein in formula (D5) and formula (D5-F) the two endocyclic bonds C³-C⁴ and C⁴-C⁵ are either single bonds or one of said two bonds is a single bond and the other bond of said two bonds is a double bond.

In formula (D6) the C² atom can be unsubstituted or monosubstituted and the C³, C⁴, C⁵ and C⁶ atoms can independently from each other be unsubstituted, mono- or disubstituted.

In formula (D5) the C² atom can be unsubstituted or monosubstituted and the C³, C⁴, and C⁵ atoms can independently from each other be unsubstituted, mono- or disubstituted.

The H atom in formula (D6) and in formula (D5) is eliminated during the deoxyfluorination reaction.

### BRIEF DESCRIPTION OF THE DRAWINGS

FIG. 1 shows the flow configuration reaction set up which was employed in the examples.

### DETAILED DESCRIPTION OF THE INVENTION

The invention relates to a deoxyfluorination. The reaction is a deoxyfluorination reaction. Both two terms deoxofluorination and deoxyfluorination are used in the prior art to describe an exchange of an oxygen atom against one or two fluorine atoms, as the case may be. In this specification these two terms are used synonymously.

In one embodiment, the subject of the invention is a method for deoxyfluorination of anyone of substrates a), b), c) and d),
by a reaction of said substrate a), b), c) or d) with the fluorination reagent SF₄ in the presence of at least 1 equiv of a base, the equiv are molar equiv based on the molar amount of substrate;
the reaction is done in a continuous flow reaction mode;
the reaction temperature is from −25 to 200° C.;
with the substrates a), b), c) and d) as defined herein.

In one embodiment, the invention relates to the method for deoxyfluorination of substrate a), with the substrates a) as defined herein.

In one embodiment, the invention relates to the method for deoxyfluorination of substrate b), with the substrates b) as defined herein.

In one embodiment, the invention relates to the method for deoxyfluorination of substrate c), with the substrates c) as defined herein.

In one embodiment, the invention relates to the method for deoxyfluorination of substrate d), with the substrates d) as defined herein.

Substrate c) can e.g. be a di-, tri- or tetra-peptide of natural alpha amino acids. The said COOH of substrate c) can in this case be the C-terminal COOH of the peptide or a COOH residue in a side chain of the peptide.

In one embodiment, the reaction is done under anhydrous conditions; preferably the residual amount of water in the reaction mixture at the beginning of the reaction is 1′000 ppm or less, more preferably 750 ppm or less, even more preferably 500 ppm or less, especially 250 ppm or less, more especially 100 ppm or less, even more especially 75 ppm or less, in particular 50 ppm or less, more in particular 25 ppm or less, even more in particular, 10 ppm or less. The reaction can be done without a solvent. Preferably, the reaction can be done without a solvent if the substrate and the product are liquid or gaseous, that is not solid, at the chosen reaction temperature.

The reaction can be done in a solvent.

The solvent is a solvent which is inert under reaction conditions, in particular inert against SF₄, HF and SOF₂ under reaction conditions. Furthermore the solvent should preferably be chosen such that the substrate and/or SF₄, preferably both, have a high solubility in the solvent.

Preferably, the solvent is selected from the group consisting of benzene, chlorobenzene toluene, methylene chloride, dichloromethane, fluorotrichloromethane, tetrahydrofuran, C₅₋₁₀ alkane, C₁₋₄ carboxylic acid C₁₋₄ alkyl ester, and C₂₋₄ alkyl C₂₋₄ alkyl ether, wherein C₅₋₁₀ alkane are preferably linear or cyclic, branched or unbranched C₅₋₁₀ alkane, such as pentane, hexane, heptane, octane, nonane and decan; more preferably linear or cyclic, branched or unbranched C₆₋₁₀ alkane.

Hexane can for example be branched or unbranched hexane or cyclohexane.

Heptane can for example be branched or unbranched heptane or methyl cyclohexane.

More preferably, the solvent is selected from the group consisting of cyclohexane, C₂₋₄ carboxylic acid C₁₋₄ alkyl ester, and C₂₋₄ alkyl C₂₋₄ alkyl ether.

In a particular embodiment, the solvent is selected from the group consisting of cyclohexane, ethyl acetate, butyl acetate, diisopropyl ether, methyl tert-butyl ether and isopropyl acetate. In a preferred particular embodiment the solvent is ethyl acetate or butyl acetate. In a more preferred particular embodiment the solvent is ethyl acetate.

Butyl acetate is preferably n-butyl acetate.

Preferably, the amount of solvent is chosen such that the concentration of the substrate in the solvent is from 0.001 to 5 M, more preferably from 0.01 to 3 M, even more preferably from 0.01 to 2 M.

Preferably, the solvent is not HF.

Preferably, the reaction is done without any addition of HF to the reaction.

Preferably, the amount of SF₄ is from 1 to 10 equiv, more preferably from 1 to 5 equiv, even more preferably from 1 to 3 equiv, especially from 1 to 2.5 equiv, the equiv being molar equiv based on the molar amount of substrate.

Preferably, in case of substrates a), b), d) and e), the amount of SF₄ is from 1 to 10 equiv, more preferably from 1 to 5 equiv, even more preferably from 1 to 3 equiv, especially from 1 to 2 equiv, the equiv being molar equiv based on the molar amount of substrate.

Preferably, in case of substrates c), the amount of SF₄ is from 1 to 10 equiv, more preferably from 1.5 to 5 equiv, even more preferably from 1.5 to 3 equiv, especially from 1.5 to 2.5 equiv, the equiv being molar equiv based on the molar amount of substrate.

If the substrate has more than one residue that are intended to react with the SF₄, then the amount of SF₄ is at least equal to the combined molar equiv of all the reacting residues in the substrate.

Preferably, the base is selected from the group consisting of pyridine, R10(R11)(R12)N, piperidine, pyridine and 1,8-Diazabicyclo[5.4.0]undec-7-ene (DBU), wherein
R10, R11 and R12 are identical or different and independently from each other selected from the group consisting of H and C₁₋₄ alkyl;
preferably R10, R11 and R12 are identical or different and independently from each other selected from the group consisting of H, methyl, ethyl, n-propyl, and isopropyl;
more preferably R10, R11 and R12 are identical or different and independently from each other ethyl or isopropyl.

In a particular embodiment, the base is triethylamine (Et₃N) or N,N-diisopropylethylamine (DIPEA).

In a more particular embodiment, the base is Et₃N.

In another more particular embodiment, the base is DIPEA.

Preferably, the amount of the base is from 1 to 11 equiv, more preferably from 1 to 6 equiv, even more preferably from 1 to 4 equiv, especially from 1 to 3 equiv, more especially from 1 to 2.5 equiv, even more especially from 1 to 2.1 equiv, the equiv being molar equiv based on the molar amount of substrate.

If the substrate has more than one residue that are intended to react with the SF₄, then the amount of base is at least equal to the combined molar equiv of all the reacting residues in the substrate.

When the reaction is done in the presence of a solvent, then the reaction temperature may be above or below the boiling point of the solvent at ambient pressure.

Lower limits of the temperature can be −25° C., −10° C., 0° C., 10° C. or 20° C.

Upper limits of the temperature can be 200° C. or 175° C.

In one embodiment of the invention, The reaction temperature can be from −25 to 200° C., preferably from −10 to 200° C., more preferably from 0 to 200° C., even more preferably from 10 to 200° C., especially from 10 to 175° C., more especially from 15 to 175° C., even more especially from 20 to 175° C.

In the continuous flow reaction mode the substrate and the SF₄ are mixed continuously in a mixing device (MIXDEV) in the presence of the base.

The continuous mixing in MIXDEV is done by continuously pumping two feeds FEED1 and FEED2, FEED1 containing the substrate and FEED2 containing SF₄, into and through MIXDEV.

Before the mixing of the substrate with SF₄ in MIXDEV, either the base is mixed with SF₄ or the base is mixed with the substrate, or the substrate is mixed with the base and SF₄ is mixed with the base, so the embodiments of the feeds FEED1 and FEED2 can be as shown in Table 6:

TABLE 6

| Embodiment | FEED1 | FEED2 |
|---|---|---|
| (i) | Substrate | Mixture of SF₄ and the base |
| (ii) | Mixture of substrate and the | SF₄ |
| | base | |
| (iii) | Mixture of substrate and the | Mixture of SF₄ and the base |
| | base | |

A preferred embodiment of the feeds in case of absence of a solvent is embodiment (ii) of Table 6.

In a particular embodiment, particularly in case of absence of a solvent, FEED1 comprises the substrate and the base and FEED2 comprises the SF₄.

In a particular embodiment, particularly in case of absence of a solvent, the substrate is mixed with the base prior to reaction with the SF₄.

In case that the reaction is done in a solvent, the embodiments of the feeds can be as shown in Table 7:

TABLE 7

| Embodiment | FEED1 | FEED2 |
|---|---|---|
| (x-i) | substrate | Mixture of SF₄ and the base |
| | | and solvent |
| (x-ii) | Mixture of substrate and | Mixture of SF₄ and the base |
| | solvent | |
| (x-iii) | Mixture of substrate and | Mixture of SF₄ and the base |
| | solvent | and solvent |
| (x-iv) | Mixture of substrate and | Mixture of SF₄ and solvent |
| | the base | |
| (x-v) | Mixture of substrate and | Mixture of SF₄ and the base |
| | the base | and solvent |
| (x-vi) | Mixture of substrate and | SF₄ |
| | the base and solvent | |
| (x-vii) | Mixture of substrate and | Mixture of SF₄ and the base |
| | the base and solvent | |
| (x-viii) | Mixture of substrate and | Mixture of SF₄ and solvent |
| | the base and solvent | |
| (x-ix) | Mixture of substrate and | Mixture of SF₄ and the base |
| | the base and solvent | and solvent |

A preferred embodiment of the feeds in case that the reaction is done in a solvent is embodiment (x-vi) of Table 7.

In a particular embodiment, FEED1 comprises substrate and base in a solvent and FEED2 comprises SF₄.

In a particular embodiment, the substrate and the base are mixed with and/or dissolved in the solvent prior to reaction with SF₄. preferably for the reaction to take place a mixture of the substrate, the base and the solvent is mixed with SF₄.

The continuous mixing in MIXDEV, that is the pumping of FEED1 and FEED2 into and through MIXDEV, is preferably done using a back pressure regulation device BPR. The BPR is located downstream from MIXDEV. The BPR provides for having a predetermined pressure in MIXDEV and for maintaining this predetermined pressure in MIXDEV at a constant level.

The reaction is preferably done at a pressure, also called reaction pressure herein, which is above the vapor pressure at the chosen reaction temperature of a mixture containing all components of the reaction mixture except for the SF₄, and/or the reaction pressure is above the vapor pressure of SF₄ at the chosen reaction temperature. The embodiment is preferred for reactions in industrial scale, so preferably SF₄ is used in liquid state.

More preferably, the reaction pressure is above the vapor pressure of the reaction mixture at the chosen reaction temperature. Typical ranges for the reaction pressure can be from 0 to 200 barg, more preferably from 0 to 150 barg, even more preferably from 0 to 100 barg.

In the absolute unit bar the reaction pressure can be from atmospheric pressure to 200 bar, more preferably from atmospheric pressure to 150 bar, even more preferably from atmospheric pressure to 100 bar.

The pressure of the reaction, that is also the pressure in MIXDEV, can be chosen and set by the BPR.

Suitable back pressure regulation devices BPR are known to the skilled person and are available on the market, such as from companies like Swagelok Company, Solon, Ohio, US, or Zaiput Flow Technologies, Scottsdale, Arizona, US. Preferably, the BPR features a small dead volume, precise pressure control and/or a large enough channel width to ensure a smooth flow without clogging.

For conveying anyone of FEED1, FEED2 and/or the reaction mixture respective pumps can be used. Depending on the components, on the pressure and on the temperature, each of FEED1, FEED2 and the reaction mixture can either be gaseous or liquid. For determining any gaseous flow rate \dot{V}_{g} and/or any liquid flow rate \dot{V}ₗ respective flow meters or mass flow controllers can be used. Pressure and temperature can be measured by respective measuring devices. All such devices are know to the skilled person.

Depending on the chosen reaction temperature the vapor pressure of SF₄ is below, equal or above the chosen pressure of the reaction.

If the vapor pressure of SF₄ is above the chosen pressure of the reaction then SF₄ can also be fed and moved through MIXDEV and any further devices driven by its own vapor pressure, so a pump may not be required in this case. But also a pump can be used to feed SF₄, especially when the vapor pressure of SF₄ is not sufficiently above the desired and chosen reaction pressure or if it is equal or even below the desired and chosen reaction pressure.

Mass flow controllers MFC can be used to determine and control and maintain any flow rate, be it gaseous or liquid of any of the components of the reaction mixture. For example a MFC can be used upstream before the MIXDEV, to determine and control and maintain the gaseous flow rate \dot{V}_{g} of SF₄.

For cleaning and especially for drying of any devices which come into contact with any of the components of the reaction mixture N₂ can be used.

MIXDEV can be any suitable device with means for combining two fluid feeds or for combining a fluid feed with a gaseous feed to provide a reaction mixture. MIXDEV can further comprise means for mixing the reaction mixture. MIXDEV can be for example a T-piece, a microreactor, a mixing device, such as a static mixing device, or any combination thereof.

In case the MIXDEV is a T-piece, which has two entrance channels, the two feeds are fed each into one of the two separate entrance channels, the two separate entrance channels combine in the T-piece into the one exit channel of the T-piece, through which the mixed feeds, that is the reaction mixture, exits.

Mixing devices such as dynamic mixing devices or static mixing devices, e.g. static mixers, are well established and widespread in all fields of chemical process technology. It is characteristically for static mixing devices, in contrast to dynamic mixing devices, that only the media to be mixed are in motion. The feeds, liquid or gaseous, are mixed by pump energy only, while the geometrically defined mixing elements in the static mixing devices remain in position. Companies such as Fluitec, Seuzachstrasse, 8413 Neftenbach, Switzerland, or Sulzer Ltd, Neuwiesenstrasse 15, 8401 Winterthur, Switzerland, are well known suppliers among others of such static mixing devices.

Preferably, the static mixing device has the form of a tube or a plate containing means that present obstacles for the flow of the reaction mixture and thereby effecting the mixing of the feeds.

The actual combining of the two feeds may be done within a mixing device, or first the two feeds are combined by a T-piece and downstream after the T-piece the reaction mixture passes through a mixing device, in this embodiment MIXDEV comprises both the T-piece and the mixing device.

Micro reactors, also called micro structured reactors, are devices in which chemical reactions take place in a confinement with typical lateral dimensions below 1 mm; the most typical form of such confinement are micro channels. A micro reactor is a continuous flow reactor. Microreactors have been successfully applied in lab, pilot and production scale. E.g. the Fraunhofer Institute for Chemical Technology ICT, Joseph-von-Fraunhofer Strasse 7, 76327 Pfinztal, Germany, or Ehrfeld Mikrotechnik GmbH, Mikroforum Ring 1, 55234 Wendelsheim, Germany, develop and offer such micro reactors.

Preferably, the micro reactor contains micro channels which are arranged in such a way as to effect the mixing of the feeds. One embodiment of a microreactor is called a split and recombine mixer.

Usually a microreactor comprises both the means for combining the two feeds and the means for mixing the obtained reaction mixture.

The reaction starts when the feeds are mixed in MIXDEV.

The time which the reaction mixture needs for passing through MIXDEV is a mixing time t_{Mix}. The reaction time is equal to or larger than t_{Mix}. If MIXDEV is a simply T piece then t_{Mix} is rather short.

The reaction mixture exiting from MIXDEV can pass through a residence device (RESDEV). RESDEV provides for additional reaction time. RESDEV is located downstream from MIXDEV.

In a particular embodiment, the reaction mixture is passed through a residence device (RESDEV) after having passed through MIXDEV.

In the case that both MIXDEV and RESDEV are used then a BPR is preferably located downstream of RESDEV.

The time which the reaction mixture needs for passing through RESDEV is a residence time t_{Res}. t_{Res} is part or the reaction time. If not RESDEV is present, t_{Res} is zero.

RESDEV has a channel through which the reaction mixture passes through, the length and the inner diameter of this channel are chosen in such a way that a desired residence time is provided for. RESDEV may for example be a tube, the tube may have the shape of a coil, herein also called reactor coil. Additional residence time means additional reaction time, in which the reaction can take place.

In the case that RESDEV is used then the reaction time is equal to or larger than the sum of t_{Mix} and t_{Res}.

Dimensional limitation of the inner diameter of MIXDEV and of RESDEV may be required primarily in connection with the use of SF₄ in gaseous state; this may for example be the case in small scale, that is for example in lab scale, reactions. In order to have an optimum of mixing in lab scale the inner diameter of MIXDEV and/or RESDEV, that is the inner diameter of any channel in MIXDEV and/or RESDEV, through which the reaction mixture passes, should be rather small, such as 2 mm or less, preferably 1 mm or less.

In a particular embodiment, the inner diameter of MIXDEV and/or RESDEV, that is the inner diameter of any channel in MIXDEV and/or RESDEV, through which the reaction mixture passes, is 2 mm or less, preferably 1 mm or less.

The handling of SF₄ in liquid state is easier to be realized on industrial scale than in the lab. When both the feed of SF₄ and the feed of the substrate are in liquid state then the mixing pertains to a liquid-liquid mixing, thereby a mixing can be effected more easily by respective and well-known mixing elements in MIXDEV than a gaseous-liquid mixing (as e.g. on lab scape).

Preferably after a BPR the product is isolated from the reaction mixture, methods and means for isolation are known to the skilled person.

In one embodiment, the reaction mixture is separated in a separation device (SEPDEV) into a gaseous stream and a liquid stream, or into a more hydrophobic and a more hydrophilic stream, as the case may be. This can be done by any means known to the skilled person for separating gas from liquid or hydrophobic components from hydrophilic components; preferably SEPDEV is a membrane separator.

In one embodiment SEPDEV separates any residual SF₄ from the reaction mixture, this separation preferably ends the reaction.

The time which the reaction mixture needs for passing through a SEPDEV is a separation time t_{Sep}. t_{Sep} is part of the reaction time. If not SEPDEV is present, t_{Sep} is zero.

A SEPDEV is located downstream after MIXDEV. When a RESDEV is used, then a SEPDEV is located downstream after RESDEV.

A SEPDEV can be located upstream before a BPR or downstream after a BPR. Preferably a SEPDEV is located downstream after a BPR,

Depending on the nature of the product, such as its boiling point or its solubility in any solvent in the reaction mixture, the product may be in the gaseous stream or in the liquid stream or in both of these two streams, likewise the product may be in the hydrophilic stream or in the hydrophobic stream or in both streams. Means for isolation of the product from the respective stream are known to the skilled person.

In a particular embodiment, the reaction mixture is passed through a separation device SEPDEV which is located downstream of the mixing device MIXDEV, the residence device RESDEV (if present) and the back pressure regulation device BPR (if present).

In one embodiment, the reaction mixture passes through SEPDEV which is a membrane separator comprising a hydrophobic membrane to separate the reaction mixture into a hydrophilic stream, which will be retained by the membrane, and a hydrophobic stream, which can pass through the membrane.

In one embodiment, the separation device SEPDEV is a membrane separator comprising a hydrophobic membrane. In this embodiment the hydrophilic stream may contain any residual amount of SF₄ and the hydrophobic stream may contain a solvent and any dissolved compounds.

In order to capture unreacted SF₄ and/or any by-products such as HF or SOF₂, anyone of the gaseous and liquid stream or both streams, or anyone of the hydrophilic and hydrophobic stream or in both streams respectively, can be quenched, for example with aqueous NaOH.

The reaction time can be defined as the time during which the substrate is in contact with the SF₄, that the time from mixing the substrate with the SF₄ until essentially all residual SF₄ in the reaction mixture is removed from the reaction mixture, that is from any substrate left in the reaction mixture, and/or is rendered inactive. SF₄ can be rendered inactive for example by reaction with aqueous NaOH. With reference to the devices the reaction time can be defined as the time during which the reaction mixture passes from the point of mixing of the SF₄ with the substrate in MIXDEV on through a RESDEV, if a RESDEV is used, and on through a SEPDEV, if a SEPDEV is used, where any residual SF₄ is separated from the reaction mixture.

In one particular embodiment, when a BPR is used, the reaction time can be defined as the time during which the reaction mixture passes from the point of mixing of the SF₄ with the substrate in MIXDEV on through a RESDEV, if a RESDEV is used, and on through the BPR, and further on through a SEPDEV, if SEPDEV is used and is located downstream after BPR, where any residual SF₄ is separated from the reaction mixture.

Preferably the reaction time is defined as the time during which the reaction mixture passes from the point of mixing of the SF₄ with the substrate in MIXDEV to a SEPDEV, where any residual SF₄ is separated from the reaction mixture.

Preferably, the reaction time is from 0.1 to 60 min, more preferable from 0.2 to 50 min, even more preferably from 0.4 to 40 min.

The reaction time is equal to or larger than the sum of t_{Mix}, t_{Res} and t_{Sep}.

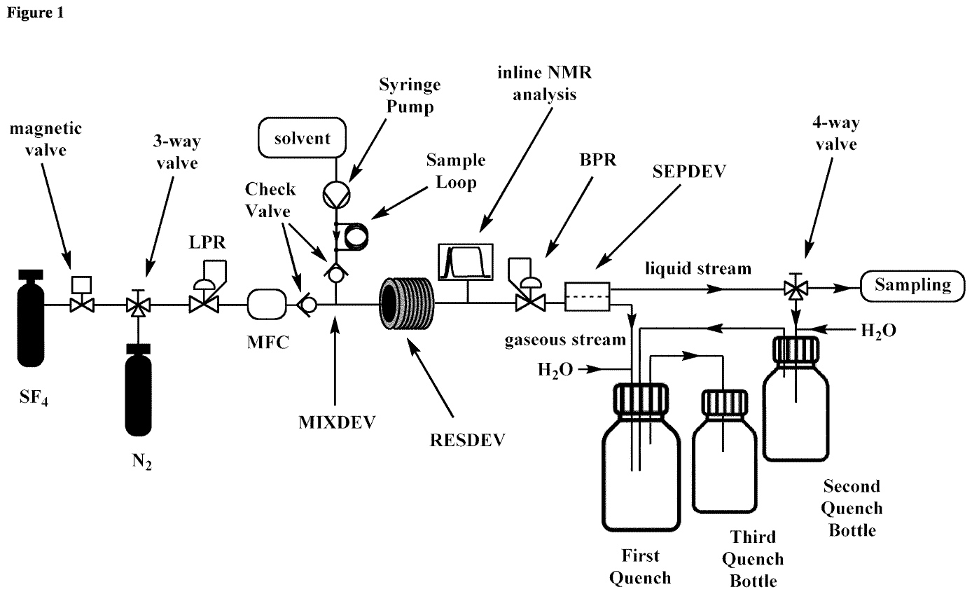

The reaction time in the set up in the examples of the (A) Flow Configuration as shown in FIG. 1 was ca. 1.1 times t_{Res}.

In the examples the RESDEV is the reactor coil. t_{Res} is calculated based on the liquid flow rate \dot{V}ₗ.

The chosen reaction temperature can be attained and maintained by respective heating or cooling of one or more of anyone of the reactants, feeds, reaction mixture, devices through which the feeds and the reaction mixture passes. In one embodiment, MIXDEV is heated or cooled and/or a RESDEV is heated or cooled to attain and maintain the chosen reaction temperature.

For pressure control, for example if any of the devices has a lower limit below which the pressure must not fall, or has an upper limit of pressure which must not be exceeded, line pressure regulators LPR or BPRs can be installed as required.

The following definitions are used:
Aryl is a carbocyclic aromatic ring or ring system, or a heterocyclic aromatic ring or ring system; a terminal aryl within the meaning of the invention is an aryl which is not substituted by another aryl.
Preferably, any heterocyclic aromatic ring can have 1, 2 or 3 identical or different endocyclic heteroatoms selected from the group consisting of N, O and S, if not defined explicitly otherwise.
Any alkyl is unbranched or branched alkyl. More specifically, C₁₋ₙ alkyl comprises unbranched or branched C₁₋ₙ alkyl with n>1.
Any alkenyl is unbranched or branched. More specifically, C₂₋ₙ alkenyl comprises unbranched or branched C₂₋ₙ alkenyl with n>2. Any alkenyl can have one or more unsaturated bonds depending on the size and connectivity of its carbon scaffold.
Alkylene is a disubstituted alkyl. Alkylene comprises unbranched or branched alkylene. More specifically, C₁₋ₙ alkylene comprises unbranched or branched C₁₋ₙ alkylene n>1.
Alkenylene is a disubstituted alkenyl. Alkenylene comprises unbranched or branched alkenylene. More specifically, C₂₋ₙ alkenylene comprises unbranched or branched C₂₋ₙ alkenylene with n>2. Any alkenylene can have one or more unsaturated bonds depending on the size and connectivity of its carbon scaffold.
Propylene is n-propylene or isopropylene.

Preferably,
substrate a) is a primary or secondary alcohol of formula (A), wherein the hydroxyl residue of the primary or secondary alcohol is exchanged against fluorine providing the (mono)fluorine compound of formula (A-F) according to Scheme A;

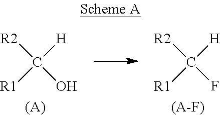

substrate b) is an aldehyde of formula (B), wherein the oxo residue of the aldehyde group of the aldehyde is exchanged against two geminal fluorine atoms providing the respective difluoro compound of formula (B-F) according to Scheme B;

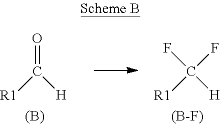

substrate c) is a carboxylic acid of formula (C), wherein the hydroxyl residue of the carboxylic acid is exchanged against fluorine providing the respective acid fluoride of formula (C-F) according to Scheme C

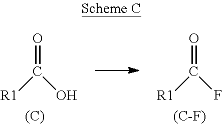

with regard to substrate d),
said unsubstituted or substituted cyclohexanone of formula (D6) or the unsubstituted or substituted cyclohexenone of formula (D6) is a compound of formula (D6-I),
said unsubstituted or substituted cyclopentanone of formula (D5) or an unsubstituted or substituted cyclopentenone of formula (D5) is a compound of formula (D5-I),
said vinyl fluoride compound of formula (D6-F) of Scheme D is a compound of formula (D6-F-I),
said vinyl fluoride compound of formula (D5-F) of Scheme D is a compound of formula (D5-F-I),
wherein the oxo residue of the keto group is exchanged against fluorine according to Scheme D-I,

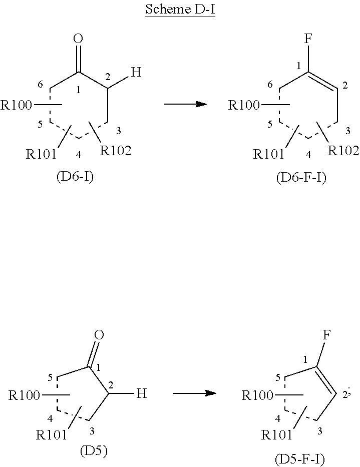

wherein in formula (D6-I) and formula (D6-F-I) the three endocyclic bonds C³-C⁴, C⁴-C⁵ and C⁵-C⁶ are either single bonds or one of said three bonds is a double bond and the other two bonds of said three bonds are single bonds, and
wherein in formula (D5-I) and formula (D5-F-I) the two endocyclic bonds C³-C⁴ and C⁴-C⁵ are either single bonds or one of said two bonds is a single bond and the other bond of said two bonds is a double bond,
or, alternatively,
substrate d) is a steroid of formula (GON) or a steroid of formula (CHOL) substituted with an oxo at C³ or at C¹⁷ of the steroid of formula (GON), or at C³ of the steroid of formula (CHOL), respectively;
wherein
R1 and R2 are identical or different and independently from each other selected from the group consisting of
   H, C₁₋₃₀ alkyl, C₂₋₃₀ alkenyl, F, Cl, Br, I, NO₂,
   O—C₁₋₁₀ alkyl, O—C₂₋₁₀ alkenyl,
   (O—CH₂—CH₂)₁₋₁₅₀—O—R6,
   (O-propylene)₁₋₁₅₀-O—R6,
   (O—CH₂—CH₂)₁₋₁₅₀—(O-propylene)₁₋₁₅₀(O—CH₂—CH₂)₀₋₁₅₀—O—R6,
   (O-propylene)₁₋₁₅₀-(O—CH₂—CH₂)₁₋₁₅₀—(O-propylene)₀₋₁₅₀-O—R6;
   C(O)H, C(O)—C₁₋₁₀ alkyl, C(O)—C₂₋₁₀ alkenyl,
   COOH, C(O)—O—C₁₋₁₀ alkyl, C(O)—O—C₂₋₁₀ alkenyl,
   C(O)—(O—CH₂—CH₂)₁₋₁₅₀—O—R6,
   C(O)—(O-propylene)₁₋₁₅₀-O—R6,
   C(O)—(O—CH₂—CH₂)₁₋₁₅₀—(O-propylene)₁₋₁₅₀(O—CH₂—CH₂)₀₋₁₅₀—O—R6,
   C(O)—(O-propylene)₁₋₁₅₀-(O—CH₂—CH₂)₁₋₁₅₀—(O-propylene)₀₋₁₅₀-O—R6;
   O—R5, S—R5, N(H)R5,
   a ring RINGALIPH,
   (Y1)-RINGALIPH,
   aryl, and
   (Y1)-aryl;
or
R1 and R2 together with the connecting C atom form a ring RINGALIPH;
R100, R101 and R102 are identical or different and independently from each other selected from the group consisting of
   oxo, H, C₁₋₃₀ alkyl, C₂₋₃₀ alkenyl,
   OH, SH, F, Cl, Br, I, NO₂,
   O—C₁₋₁₀ alkyl, O—C₂₋₁₀ alkenyl,
   (O—CH₂—CH₂)₁₋₁₅₀—O—R6,
   (O-propylene)₁₋₁₅₀-O—R6,
   (O—CH₂—CH₂)₁₋₁₅₀—(O-propylene)₁₋₁₅₀(O—CH₂—CH₂)₀₋₁₅₀—O—R6,
   (O-propylene)₁₋₁₅₀-(O—CH₂—CH₂)₁₋₁₅₀—(O-propylene)₀₋₁₅₀-O—R6,
   C(O)H, C(O)—C₁₋₁₀ alkyl, C(O)—C₂₋₁₀ alkenyl,
   COOH, C(O)—O—C₁₋₁₀ alkyl, C(O)—O—C₂₋₁₀ alkenyl,
   C(O)—(O—CH₂—CH₂)₁₋₁₅₀—O—R6,
   C(O)—(O-propylene)₁₋₁₅₀-O—R6,
   C(O)—(O—CH₂—CH₂)₁₋₁₅₀—(O-propylene)₁₋₁₅₀(O—CH₂—CH₂)₀₋₁₅₀—O—R6,
   C(O)—(O-propylene)₁₋₁₅₀-(O—CH₂—CH₂)₁₋₁₅₀—(O-propylene)₀₋₁₅₀-O—R6,
   O—R5, S—R5, N(H)R5,
   aryl, and
   (Y1)-aryl;
and wherein
any mentioned RINGALIPH is, independently from any other RINGALIPH,
   a 4, 5 or 6 membered saturated or unsaturated, non-aromatic carbocyclic or heterocyclic residue, the heterocyclic residue containing 1 or 2 identical or different endocyclic heteroatoms Y2,
   a steroid residue, or
   a bicyclic spiro residue,
   each ring of the bicyclic spiro residue is independently from the other ring a 4, 5 or 6 membered ring,
   in case of a 4 membered ring of the bicyclic spiro residue the ring contains 0 or 1 endocyclic heteroatom Y2,
   in case of a 5 or 6 membered ring of the bicyclic spiro residue the ring contains 0, 1 or 2 identical or different endocyclic heteroatoms Y2;
and
RINGALIPH is unsubstituted or substituted by one or more substituents independently from each other selected from the group consisting of
   oxo, C₁₋₃₀ alkyl, C₂₋₃₀ alkenyl, OH, SH, F, Cl, Br, I, NO₂,
   O—C₁₋₁₀ alkyl, O—C₂₋₁₀ alkenyl,
   (O—CH₂—CH₂)₁₋₁₅₀—O—R6,
   (O-propylene)₁₋₁₅₀-O—R6,
   (O—CH₂—CH₂)₁₋₁₅₀—(O-propylene)₁₋₁₅₀(O—CH₂—CH₂)₀₋₁₅₀—O—R6,
   (O-propylene)₁₋₁₅₀-(O—CH₂—CH₂)₁₋₁₅₀—(O-propylene)₀₋₁₅₀-O—R6,
   C(O)H, C(O)—C₁₋₁₀ alkyl, C(O)—C₂₋₁₀ alkenyl,
   COOH, C(O)—O—C₁₋₁₀ alkyl, C(O)—O—C₂₋₁₀ alkenyl,
   C(O)—(O—CH₂—CH₂)₁₋₁₅₀—O—R6,
   C(O)—(O-propylene)₁₋₁₅₀-O—R6,
   C(O)—(O—CH₂—CH₂)₁₋₁₅₀—(O-propylene)₁₋₁₅₀(O—CH₂—CH₂)₀₋₁₅₀—O—R6,
   C(O)—(O-propylene)₁₋₁₅₀-(O—CH₂—CH₂)₁₋₁₅₀—(O-propylene)₀₋₁₅₀-O—R6,
   O—R5, S—R5, N(H)R5,
   aryl, and
   (Y1)-aryl;
any mentioned aryl is, independently from any other aryl,
   a 5 or 6 membered monocyclic aryl residue,
   a bicyclic aryl residue formed by a 5 or 6 membered ring fused with another 5 or 6 membered ring, or
   an aryl residue wherein a 5 or 6 membered aromatic ring is fused with a 5 or 6 membered non-aromatic ring, said aryl residue being connected with either said aromatic ring or with said non-aromatic ring to the C atom of formula (A),
   any 5 membered ring in the aryl contains 0, 1 or 2 identical or different endocyclic heteroatoms Y2,
   any 6 membered ring in the aryl contains 0, 1, 2 or 3 identical or different endocyclic heteroatoms Y2;
and
aryl is unsubstituted or substituted by one or more identical or different substituents selected from the group consisting of
   C₁₋₁₀alkyl, C₂₋₁₀ alkenyl, F, Cl, Br, I, NO₂,
   O—C₁₋₁₀ alkyl, O—C₂₋₁₀ alkenyl,
   (O—CH₂—CH₂)₁₋₁₅₀—O—R6,
   (O-propylene)₁₋₁₅₀-O—R6,
   (O—CH₂—CH₂)₁₋₁₅₀—(O-propylene)₁₋₁₅₀(O—CH₂—CH₂)₀₋₁₅₀—O—R6,
   (O-propylene)₁₋₁₅₀-(O—CH₂—CH₂)₁₋₁₅₀—(O-propylene)₀₋₁₅₀-O—R6,
   C(O)H, C(O)—C₁₋₁₀ alkyl, C(O)—C₂₋₁₀ alkenyl,
   COOH, C(O)—O—C₁₋₁₀ alkyl, C(O)—O—C₂₋₁₀ alkenyl,
   C(O)—(O—CH₂—CH₂)₁₋₁₅₀—O—R6,
   C(O)—(O-propylene)₁₋₁₅₀-O—R6,
   C(O)—(O—CH₂—CH₂)₁₋₁₅₀—(O-propylene)₁₋₁₅₀-(O—CH₂—CH₂)₀₋₁₅₀—O—R6,
   C(O)—(O-propylene)₁₋₁₅₀-(O—CH₂—CH₂)₁₋₁₅₀—(O-propylene)₀₋₁₅₀-O—R6;
   O—R5, S—R5, N(H)R5,
   a terminal aryl, and
   (Y1)-aryl which is a terminal aryl;
any mentioned heteroatom Y2 is, independently from any other Y2, selected from the group consisting of O, S, and saturated or unsaturated N, said saturated N is unsubstituted or substituted by R5;
any mentioned Y1 is, independently from any other Y1, a connecting group selected from the group consisting of
   O, C₁₋₆ alkylene, C(O),
   C(O)—C₁₋₆ alkylene-C(O),
   (O—CH₂—CH₂)₁₋₁₅₀—O,
   (O-propylene)₁₋₁₅₀-O,
   (O—CH₂—CH₂)₁₋₁₅₀—(O-propylene)₁₋₁₅₀-(O—CH₂—CH₂)₀₋₁₅₀—O,
   (O-propylene)₁₋₁₅₀-(O—CH₂—CH₂)₁₋₁₅₀—(O-propylene)₀₋₁₅₀-O;
   (O—CH₂—CH₂)₁₋₁₅₀—O—C(O)—C₁₋₆ alkylene-C(O),
   (O—CH₂—CH₂)₁₋₁₅₀—O—C(O)—C₁₋₆ alkylene-C(O)—O,
   (O—CH₂—CH₂)₁₋₁₅₀—(O-propylene)₁₋₁₅₀(O—CH₂—CH₂)₀₋₁₅₀—O—C(O)—C₁₋₆ alkylene-C(O),
   (O-propylene)₁₋₁₅₀-O—C(O)—C₁₋₆ alkylene-C(O),
   (O-propylene)₁₋₁₅₀-O—C(O)—C₁₋₆ alkylene-C(O)—O,
   (O-propylene)₁₋₁₅₀-(O—CH₂—CH₂)₁₋₁₅₀—(O-propylene)₀₋₁₅₀-O—C(O)—C₁₋₆ alkylene-C(O),
   C(O)—C₁₋₆ alkylene-C(O)—(O—CH₂—CH₂)₁₋₁₅₀—O,
   C(O)—C₁₋₆ alkylene-C(O)—(O-propylene)₁₋₁₅₀-O,
   C(O)—C₁₋₆ alkylene-C(O)—(O—CH₂—CH₂)₁₋₁₅₀—(O-propylene)₁₋₁₅₀(O—CH₂—CH₂)₀₋₁₅₀—O,
   C(O)—C₁₋₆ alkylene-C(O)—(O-propylene)₁₋₁₅₀-(O—CH₂—CH₂)₁₋₁₅₀—(O-propylene)₀₋₁₅₀-O,
   C(O)—O—C₁₋₆ alkylene-O—C(O), O—C(O)—C₁₋₆ alkylene-C(O)—O,
   O—C(O)—C₁₋₆ alkylene-C(O)—(O—CH₂—CH₂)₁₋₁₅₀—O, and
   O—C(O)—C₁₋₆ alkylene-C(O)—(O-propylene)₁₋₁₅₀-O;
any mentioned C₁₋₃₀ alkyl and any mentioned C₂₋₃₀ alkenyl is, independently from any other C₁₋₃₀ alkyl and C₂₋₃₀ alkenyl respectively, unsubstituted or substituted by 1, 2, 3, 4, 5, or 6 identical of different substituents selected from the group consisting of oxo, OH, SH, F, Cl, Br, I, NO₂,
   O—C₁₋₁₀ alkyl, O—C₂₋₁₀ alkenyl,
   (O—CH₂—CH₂)₁₋₁₅₀—O—R6,
   (O-propylene)₁₋₁₅₀-O—R6,
   (O—CH₂—CH₂)₁₋₁₅₀—(O-propylene)₁₋₁₅₀(O—CH₂—CH₂)₀₋₁₅₀—O—R6,
   (O-propylene)₁₋₁₅₀-(O—CH₂—CH₂)₁₋₁₅₀—(O-propylene)₀₋₁₅₀-O—R6,
   C(O)H, C(O)—C₁₋₁₀ alkyl, C(O)—C₂₋₁₀ alkenyl,
   COOH, C(O)—O—C₁₋₁₀ alkyl, C(O)—O—C₂₋₁₀ alkenyl,
   C(O)—(O—CH₂—CH₂)₁₋₁₅₀—O—R6,
   C(O)—(O-propylene)₁₋₁₅₀-O—R6,
   C(O)—(O—CH₂—CH₂)₁₋₁₅₀—(O-propylene)₁₋₁₅₀(O—CH₂—CH₂)₀₋₁₅₀—O—R6,
   C(O)—(O-propylene)₁₋₁₅₀-(O—CH₂—CH₂)₁₋₁₅₀—(O-propylene)₀₋₁₅₀-O—R6,
   O—R5, S—R5, N(H)R5,
   aryl, and
   (Y1)-aryl;
any mentioned C₁₋₁₀ alkyl and any mentioned C₂₋₁₀ alkenyl is, independently from any other C₁₋₁₀alkyl and C₂₋₁₀ alkenyl, unsubstituted or substituted by 1, 2, 3, 4, 5, or 6 identical or different substituents selected from a group consisting of oxo, OH, SH, F, Cl, Br, I, NO₂,
   O—C₁₋₉ alkyl, O—C₂₋₉ alkenyl,
   (O—CH₂—CH₂)₁₋₁₅₀—O—R6,
   (O-propylene)₁₋₁₅₀-O—R6,
   (O—CH₂—CH₂)₁₋₁₅₀—(O-propylene)₁₋₁₅₀(O—CH₂—CH₂)₀₋₁₅₀—O—R6,
   (O-propylene)₁₋₁₅₀-(O—CH₂—CH₂)₁₋₁₅₀—(O-propylene)₀₋₁₅₀-O—R6
   C(O)H, C(O)—C₁₋₉ alkyl, C(O)—C₂₋₉ alkenyl,
   COOH, C(O)—O—C₁₋₉ alkyl, C(O)—O—C₂₋₉ alkenyl,
   C(O)—(O—CH₂—CH₂)₁₋₁₅₀—O—R6,
   C(O)—(O-propylene)₁₋₁₅₀-O—R6,
   C(O)—(O—CH₂—CH₂)₁₋₁₅₀—(O-propylene)₁₋₁₅₀(O—CH₂—CH₂)₀₋₁₅₀—O—R6,
   C(O)—(O-propylene)₁₋₁₅₀-(O—CH₂—CH₂)₁₋₁₅₀—(O-propylene)₀₋₁₅₀-O—R6,
   O—R5, S—R5, and N(H)R5;
any mentioned R5 is, independently from any other R5, a protecting group for protecting OH, SH or NH₂;
any mentioned R6 is, independently from any other R6, H or R5.
Preferably, RINGALIPH is unsubstituted or substituted by 1, 2, 3, 4, 5 or 6, more preferably by 1, 2, 3, 4 or 5, even more preferably by 1, 2, 3 or 4 substituents.
Preferably, substrate d) is a unsubstituted or substituted cyclohexanone of formula (D6-I) or a unsubstituted or substituted cyclopentanone of formula (D5-I);

more preferably, substrate d) is a unsubstituted or substituted cyclohexanone of formula (D6-I).

In formula (D6-I) the C² atom can be unsubstituted or monosubstituted and the C³, C⁴, C⁵ and C⁶ atoms can independently from each other be unsubstituted, mono- or disubstituted. In formula (D5-I) the C² atom can be unsubstituted or monosubstituted and the C³, C⁴, and C⁵ atoms can independently from each other be unsubstituted, mono- or disubstituted. The H atom in formula (D6-I) and in formula (D5-I) is eliminated during the deoxyfluorination reaction.

Preferably, said steroid residue is a steroid residue of formula (GON), which has the tetracyclic scaffold of gonan with the fours rings A, B, C and D and the numbering of the endocyclic C atoms as displayed in formula (GON);

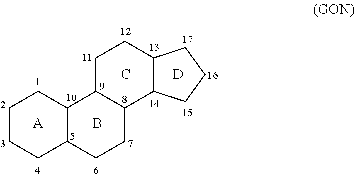

wherein
1, 2, 3, 4, 5 or 6 of the endocyclic bonds can be unsaturated;
anyone of the endocyclic C atoms independently from each other is unsubstituted or substituted by 1 or 2 substituents independently from each other selected from the group consisting of
   C₁₋₃₀ alkyl, C₂₋₃₀ alkenyl,
   OH, SH, F, Cl, Br, I, NO₂,
   O—C₁₋₁₀ alkyl, O—C₂₋₁₀ alkenyl,
   (O—CH₂—CH₂)₁₋₁₅₀—O—R6,
   (O-propylene)₁₋₁₅₀-O—R6,
   (O—CH₂—CH₂)₁₋₁₅₀—(O-propylene)₁₋₁₅₀(O—CH₂—CH₂)₀₋₁₅₀—O—R6,
   (O-propylene)₁₋₁₅₀-(O—CH₂—CH₂)₁₋₁₅₀—(O-propylene)₀₋₁₅₀-O—R6,
   C(O)H, C(O)—C₁₋₁₀ alkyl, C(O)—C₂₋₁₀ alkenyl,
   COOH, C(O)—O—C₁₋₁₀ alkyl, C(O)—O—C₂₋₁₀ alkenyl,
   C(O)—(O—CH₂—CH₂)₁₋₁₅₀—O—R6,
   C(O)—(O-propylene)₁₋₁₅₀-O—R6,
   C(O)—(O—CH₂—CH₂)₁₋₁₅₀—(O-propylene)₁₋₁₅₀(O—CH₂—CH₂)₀₋₁₅₀—O—R6,
   C(O)—(O-propylene)₁₋₁₅₀-(O—CH₂—CH₂)₁₋₁₅₀—(O-propylene)₀₋₁₅₀-O—R6,
   O—R5, S—R5, N(H)R5,
   aryl, and
   (Y1)-aryl;

wherein R5, R6, C₁₋₃₀ alkyl, C₂₋₃₀ alkenyl, C₁₋₁₀ alkyl, C₂₋₁₀ alkenyl, Y1 and aryl are as defined herein.
In an embodiment, said steroid residue is a steroid residue of formula (CHOL) which has the tetracyclic scaffold of cholestane with the fours rings A, B, C and D, with the exocyclic C¹⁸ and C¹⁹ atoms, with the isoprene scaffold with the C²⁰ to C²⁷ atoms and with the IUPAC numbering as displayed in formula (CHOL);

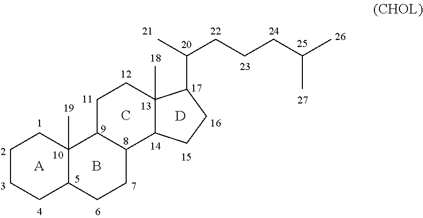

wherein
1, 2, 3, 4, 5 or 6 of the endocyclic bonds can be unsaturated;
anyone of the endocyclic C atoms independently from each other is unsubstituted or substituted by 1 or 2 substituents, independently from each other selected from the group consisting of
   C₁₋₃₀ alkyl, C₂₋₃₀ alkenyl
   OH, SH, F, Cl, Br, I, NO₂,
   O—C₁₋₁₀ alkyl, O—C₂₋₁₀ alkenyl,
   (O—CH₂—CH₂)₁₋₁₅₀—O—R6,
   (O-propylene)₁₋₁₅₀-O—R6,
   (O—CH₂—CH₂)₁₋₁₅₀—(O-propylene)₁₋₁₅₀(O—CH₂—CH₂)₀₋₁₅₀—O—R6,
   (O-propylene)₁₋₁₅₀-(O—CH₂—CH₂)₁₋₁₅₀—(O-propylene)₀₋₁₅₀-O—R6,
   C(O)H, C(O)—C₁₋₁₀ alkyl, C(O)—C₂₋₁₀ alkenyl,
   COOH, C(O)—O—C₁₋₁₀ alkyl, C(O)—O—C₂₋₁₀ alkenyl,
   C(O)—(O—CH₂—CH₂)₁₋₁₅₀—O—R6,
   C(O)—(O-propylene)₁₋₁₅₀-O—R6,
   C(O)—(O—CH₂—CH₂)₁₋₁₅₀—(O-propylene)₁₋₁₅₀(O—CH₂—CH₂)₀₋₁₅₀—O—R6,
   C(O)—(O-propylene)₁₋₁₅₀-(O—CH₂—CH₂)₁₋₁₅₀—(O-propylene)₀₋₁₅₀-O—R6,
   O—R5, S—R5, N(H)R5,
   aryl, and
   (Y1)-aryl;
wherein R5, R6, C¹⁻³⁰ alkyl, C₂₋₃₀ alkenyl, C₁₋₁₀ alkyl, C₂₋₁₀ alkenyl, Y1 and aryl are defined as herein.
More preferably in said steroid residue, in said steroid residue of formula (GON) and in said steroid residue of formula (CHOL),
one or more of the endocyclic bonds 1-2, 4-5, 5-6, 6-7 and 8-9 can be unsaturated;
ring A can be an aromatic ring;
the endocyclic C atoms 3, 4, 6, 7, 9, 10, 11, 13, 12, 14, 16 and 17 are independently from each other unsubstituted or substituted by 1 or 2 substituents selected from the group consisting of
   C₁₋₁₀ alkyl, C₂₋₁₀ alkenyl,
   OH, F, Cl, Br, I, NO₂,
   O—C₁₋₁₀ alkyl, O—C₂₋₁₀ alkenyl,
   C(O)H, C(O)—C₁₋₁₀ alkyl, C(O)—C₂₋₁₀ alkenyl,
   COOH, C(O)—O—C₁₋₁₀ alkyl, C(O)—O—C₂₋₁₀ alkenyl,
   O—R5, N(H)R5,
   aryl, and
   (Y1)-aryl;

wherein R5, C₁₋₁₀ alkyl, C₂₋₁₀ alkenyl, Y1 and aryl are as defined herein.

R5 is a protecting group for protecting OH, SH or NH₂, which is stable under reaction conditions, such groups are known to the skilled person.

Preferably,
if OH is to be protected then R5 is Fmoc, Boc, benzoyl, Cbz, C(O)—C₁₋₁₀ alkyl, TBDMS (tert-Butyldimethylsilyl), triisopropylsilyl, PNB (p-Nitrobenzyl), ONB (o-Nitrobenzyl), Bn (Benzyl), Al (Allyl), or tBu (tert-Butyl),
   more preferably Fmoc, Boc, benzoyl, Cbz, C(O)—C₁₋₆ alkyl, PNB (p-Nitrobenzyl), ONB (o-Nitrobenzyl), Bn (Benzyl), Al (Allyl), or tBu (tert-Butyl),
      even more preferably Fmoc, Boc, benzoyl, Cbz, C(O)—C₁₃ alkyl, or Bn (Benzyl);
if NH₂ is to be protected then R5 is Fmoc, Boc, benzoyl, Cbz, C(O)—C₁₋₁₀ alkyl, Bn (Benzyl), or Alloc (Allyloxycarbonyl),
   more preferably Fmoc, Boc, benzoyl, Cbz, C(O)—C₁₋₆ alkyl, Bn (Benzyl), or Alloc (Allyloxycarbonyl);
if SH is to be protected then R5 is Fmoc, Boc, benzoyl, Cbz, C₁₋₂ alkyl, C(O)—C₁₋₁₀ alkyl, Bn (Benzyl), Meb (p-Methylbenzyl), Acm (Acetamidomethyl), or Trt,
   more preferably Fmoc, Boc, benzoyl, Cbz, C₁₋₂ alkyl, C(O)—C₁₋₆ alkyl, Bn (Benzyl),
   Meb (p-Methylbenzyl), or Acm (Acetamidomethyl).

In particular, if OH is to be protected then R5 is Boc, benzoyl, Cbz, Fmoc, C(O)—C₁₋₆ alkyl or Bn (Benzyl).

Said di-, tri- or tetra-peptide can contain any combination of natural alpha amino acids. Examples of side chains of said natural alpha amino acids are selected from the group H (Gly, G), H₃C (Ala, A), (H₃C)₂CH (Val, V), CH₃CH₂CH(CH₃) (Ile, I), (CH₃)₂CHCH₂ (Leu, L), C₆H₅CH₂ (Phe, F), HOCH₂ (Ser, S), CH₃CHOH (Thr, T), H₂NC(O)CH₂ (Asn, N), H₂NC(O)CH₂CH₂ (Gln, Q), HSCH₂ (Cys, C), CH₃SCH₂CH₂ (Met, M),

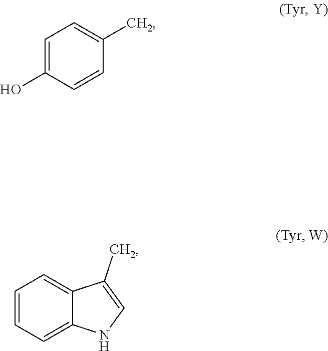

HO(O)CCH₂ (Asp, D), HO(O)CCH₂CH₂ (Glu, E),

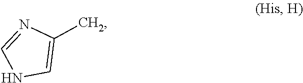

H₂N(CH₂)₃CH₂ (Lys, K), and H₂NC(NH)NH(CH₂)₂CH₂ (Arg, R); in brackets are the three- and one-letter code of the respective naturally occurring alpha amino acid.

Preferably, any functional residue contained in said di-, tri- or tetra-peptide which could react under the deoxyfluorination reaction conditions and which is not supposed to react, is protected. Such protection is done using a protecting group known to the skilled person. Such protection serves the purpose to render such functional residue inert under reaction conditions. Suitable protecting groups of functional residues, such as OH, NH₂ or SH, contained in said di-, tri- or tetra-peptide can for example be R5; for other functional residues contained in said di-, tri- or tetra-peptide protecting groups can for example be:
if said functional residue is a guanidine residue (NHC(NH)NH₂, such as in Arg): Boc, or Pbf (2,2,4,6,7-Pentamethyl-2,3-dihydrobenzofuran-5-sulfonyl);
if said functional residue is an amide residue (C(O)NH₂, such as in Asn or Gln): Xan (9-Xanthenyl), or Trt (Trityl);
if said functional residue is an imidazole residue (such as in His): Tos (Tosyl), Bom (Benzyloxymethyl), Trt, or Boc;
if said functional residue is an carboxylic acid residue (such as in Asp or Glu): tBu, Trt, Dmb (2,4-Dimethoxybenzyl), Fm (9-Fluorenylmethyl), or Bn;
if said functional residue is an indole residue (such as in Trp): For (Formyl), or Boc, preferably Boc;
if said functional residue is a methylthio residue (SCH₃, such as in Met): it can be oxidized to the sulfoxide before or during the deoxofluorination reaction, and reduced after the deoxofluorination reaction.

Preferably, in case of substrate b), R1 and R2 are not O—R5, S—R5, or N(H)R5.

Preferably, in case of substrate c), R1 and R2 are not O—R5, S—R5, or N(H)R5.
More preferably, in case of both substrates b) and c), R1 and R2 are not O—R5, S—R5, or N(H)R5.

Preferably,
R1 and R2 are identical or different and independently from each other selected from the group consisting of
   H, C₁₋₂₂ alkyl, C₂₋₂₂ alkenyl, F, Cl, Br, I,
   O—C₁₋₆ alkyl, O—C₂₋₆ alkenyl,
   C(O)H, C(O)—C₁₋₆ alkyl, C(O)—C₂₋₆ alkenyl,
   COOH, C(O)—O—C₁₋₆ alkyl, C(O)—O—C₂₋₆ alkenyl,
   O—R5, S—R5, N(H)R5,
   a ring RINGALIPH,
   (Y1)-RINGALIPH,
   aryl, and
   (Y1)-aryl;
or
R1 and R2 together with the connecting C atom form a ring RINGALIPH;
R100, R101 and R102 are identical or different and independently from each other selected from the group consisting of
   oxo, H, C₁₋₂₂ alkyl, C₂₋₂₂ alkenyl,
   OH, F, Cl, Br, I, NO₂,
   O—C₁₋₆ alkyl, O—C₂₋₆ alkenyl,
   C(O)H, C(O)—C₁₋₆ alkyl, C(O)—C₂₋₆ alkenyl,
   COOH, C(O)—O—C₁₋₆ alkyl, C(O)—O—C₂₋₆ alkenyl,
   O—R5, N(H)R5,
   aryl, and
   (Y1)-aryl;
and wherein
any mentioned RINGALIPH is, independently from any other RINGALIPH,
   a 4, 5 or 6 membered saturated or unsaturated, non-aromatic carbocycle or heterocyclic residue, the heterocyclic residue containing 1 or 2 identical or different endocyclic heteroatoms Y2, or
   a bicyclic spiro residue,
   each ring of the bicyclic spiro residue is independently from the other ring a 5 or 6 membered ring,
   in case of a 5 or 6 membered ring of the bicyclic spiro residue the ring contains 0, 1 or 2 identical or different endocyclic heteroatoms Y2;
and
RINGALIPH is unsubstituted or substituted by one or more substituents independently from each other selected from the group consisting of
   oxo, C₁₋₂₂ alkyl, C₂₋₂₂ alkenyl, OH, F, Cl, Br, I, NO₂,
   O—C₁₋₆ alkyl, O—C₂₋₆ alkenyl,
   C(O)H, C(O)—C₁₋₆ alkyl, C(O)—C₂₋₆ alkenyl,
   COOH, C(O)—O—C₁₋₆ alkyl, C(O)—O—C₂₋₆ alkenyl,
   O—R5, N(H)R5,
   aryl, and
   (Y1)-aryl;
any mentioned aryl is, independently from any other aryl,
   a 5 or 6 membered monocyclic aryl residue,
   a bicyclic aryl residue formed by a 5 or 6 membered ring fused with a 6 membered ring, or
   an aryl residue wherein a 5 or 6 membered aromatic ring is fused with a 5 or 6 membered non-aromatic ring, said aryl residue being connected with either said aromatic ring or with said non-aromatic ring to the C atom of formula (A),
any 5 membered ring in the aryl contains 0, 1 or 2 identical or different endocyclic heteroatoms Y2,
any 6 membered ring in the aryl contains 0, 1, 2 or 3 identical or different endocyclic heteroatoms Y2;
and
aryl is unsubstituted or substituted by one or more identical or different substituents selected from the group consisting of
   C₁₋₆ alkyl, C₂₋₆ alkenyl, F, Cl, Br, I, NO₂,
   O—C₁₋₆ alkyl, O—C₂₋₆ alkenyl,
   C(O)H, C(O)—C₁₋₆ alkyl, C(O)—C₂₋₆ alkenyl,
   COOH, C(O)—O—C₁₋₆ alkyl, C(O)—O—C₂₋₆ alkenyl,
   O—R5, N(H)R5,
   a terminal aryl, and
   (Y1)-aryl which is a terminal aryl;
any mentioned heteroatom Y2 is, independently from any other Y2, selected from the group consisting of 0, S, and saturated or unsaturated N, said saturated N is unsubstituted or substituted by R5;
any mentioned Y1 is, independently from any other Y1, a connecting group selected from the group consisting of
   O, C₁₋₆ alkylene, C(O),
   (O—CH₂—CH₂)₁₋₂₀—O,
   (O-propylene)₁₋₂₀-O,
   (O—CH₂—CH₂)₁₋₂₀—O—C(O)—C₁₋₆ alkylene-C(O)—O,
   (O-propylene)₁₋₂₀-O—C(O)—C₁₋₆ alkylene-C(O)—O,
   O—C(O)—C₁₋₆ alkylene-C(O)—(O—CH₂—CH₂)₁₋₂₀—O, and
   O—C(O)—C₁₋₆ alkylene-C(O)—(O-propylene)₁₋₂₀-O;
any mentioned C₁₋₂₂ alkyl and any mentioned C₂₋₂₂ alkenyl is, independently from any other C₁₋₂₂ alkyl and C₂₋₂₂ alkenyl respectively, unsubstituted or substituted by 1, 2, 3, 4, 5, or 6 identical of different substituents selected from the group consisting of oxo, OH, F, Cl, Br, I, NO₂,
   O—C₁₋₆ alkyl, O—C₂₋₆ alkenyl,
   C(O)H, C(O)—C₁₋₆ alkyl, C(O)—C₂₋₆ alkenyl,
   COOH, C(O)—O—C₁₋₆ alkyl, C(O)—O—C₂₋₆ alkenyl,
   O—R5, S—R5, N(H)R5,
   aryl, and
   (Y1)-aryl;
any mentioned C₁₋₆ alkyl and any mentioned C₂₋₆ alkenyl is, independently from any other C₁. 6 alkyl and C₂₋₆ alkenyl, unsubstituted or substituted by 1, 2, 3, 4, 5, or 6 identical or different substituents selected from a group consisting of
   oxo, OH, F, Cl, Br, I, NO₂,
   O—C₁₋₄ alkyl, O—C₂₋₃ alkenyl,
   C(O)H, C(O)—C₁₋₄ alkyl, C(O)—C₂₋₃ alkenyl,
   COOH, C(O)—O—C₁₋₄ alkyl, C(O)—O—C₂₋₃ alkenyl,
   O—R5, S—R5, and N(H)R5.

More preferably,
R1 and R2 are identical or different and independently from each other selected from the group consisting of
   H, C₁₋₂₂ alkyl, C₂₋₂₂ alkenyl, F, Cl, Br, I,
   O—C₁₋₆ alkyl, O—C₂₋₃ alkenyl,
   C(O)H, C(O)—C₁₋₆ alkyl, C(O)—C₂₋₃ alkenyl,
   COOH, C(O)—O—C₁₋₆ alkyl, C(O)—O—C₂₋₃ alkenyl,
   O—R5, S—R5, N(H)R5,
   a ring RINGALIPH,
   (Y1)-RINGALIPH,
   aryl, and
   (Y1)-aryl;
or
R1 and R2 together with the connecting C atom form a ring RINGALIPH;
R100, R101 and R102 are identical or different and independently from each other selected from the group consisting of
   oxo, H, C₁₋₂₂ alkyl, C₂₋₂₂ alkenyl,
   OH, F, Cl, Br, I, NO₂,
   O—C₁₋₆ alkyl, O—C₂₋₃ alkenyl,
   C(O)H, C(O)—C₁₋₆ alkyl, C(O)—C₂₋₃ alkenyl,
   COOH, C(O)—O—C₁₋₆ alkyl, C(O)—O—C₂₋₃ alkenyl,
   O—R5, N(H)R5,
   aryl, and
   (Y1)-aryl;
and wherein
any mentioned RINGALIPH is, independently from any other RINGALIPH,
   a 4, 5 or 6 membered saturated or unsaturated, non-aromatic carbocycle or heterocyclic residue, the heterocyclic residue containing 1 or 2 identical or different endocyclic heteroatoms Y2;
and
RINGALIPH is unsubstituted or substituted by one or more substituents independently from each other selected from the group consisting of
   oxo, C₁₋₂₂ alkyl, C₂₋₂₂ alkenyl, OH, F, Cl, Br, I, NO₂,
   O—C₁₋₆ alkyl, O—C₂₋₃ alkenyl,
   C(O)H, C(O)—C₁₋₆ alkyl, C(O)—C₂₋₃ alkenyl,
   COOH, C(O)—O—C₁₋₆ alkyl, C(O)—O—C₂₋₃ alkenyl,
   O—R5, N(H)R5,
   aryl, and
   (Y1)-aryl;
any mentioned aryl is, independently from any other aryl,
a 5 or 6 membered monocyclic aryl residue,
   a bicyclic aryl residue formed by a 5 or 6 membered ring fused with a 6 membered ring, or
   an aryl residue wherein a 5 or 6 membered aromatic ring is fused with a 5 or 6 membered non-aromatic ring, said aryl residue being connected with either said aromatic ring or with said non-aromatic ring to the C atom of formula (A),
any 5 membered ring in the aryl residue contains 0, 1 or 2 identical or different endocyclic heteroatoms Y2,
any 6 membered ring in the aryl residue contains 0, 1, 2 or 3 identical or different endocyclic heteroatoms Y2;
and
aryl is unsubstituted or substituted by one or more identical or different substituents selected from the group consisting of
   C₁₋₆ alkyl, C₂₋₃ alkenyl, F, Cl, Br, I, NO₂,
   O—C₁₋₆ alkyl, O—C₂₋₃ alkenyl,
   C(O)H, C(O)—C₁₋₆ alkyl, C(O)—C₂₋₃ alkenyl,
   COOH, C(O)—O—C₁₋₆ alkyl, C(O)—O—C₂₋₃ alkenyl,
   O—R5, N(H)R5;
any mentioned heteroatom Y2 is, independently from any other Y2, selected from the group consisting of 0, S, and saturated or unsaturated N, said saturated N is unsubstituted or substituted by R5;
any mentioned Y1 is, independently from any other Y1, a connecting group selected from the group consisting of
   O, C₁₋₆ alkylene, C(O),
   (O—CH₂—CH₂)₁₋₁₀—O,
   (O-propylene)₁₋₁₀-O,
   (O—CH₂—CH₂)₁₋₁₀—O—C(O)—C₁₋₆ alkylene-C(O)—O,
   (O-propylene)₁₋₁₀-O—C(O)—C₁₋₆ alkylene-C(O)—O,
   O—C(O)—C₁₋₆ alkylene-C(O)—(O—CH₂—CH₂)₁₋₁₀—O, and
   O—C(O)—C₁₋₆ alkylene-C(O)—(O-propylene)₁₋₁₀-O;
any mentioned C₁₋₂₂ alkyl and any mentioned C₂₋₂₂ alkenyl is, independently from any other C₁₋₂₂ alkyl and C₂₋₂₂ alkenyl respectively, unsubstituted or substituted by 1, 2, 3, 4, 5, or 6 identical of different substituents selected from the group consisting of oxo, OH, F, Cl, Br, I, NO₂,
   O—C₁₋₆ alkyl, O—C₂₋₃ alkenyl,
   C(O)H, C(O)—C₁₋₆ alkyl, C(O)—C₂₋₃ alkenyl,
   COOH, C(O)—O—C₁₋₆ alkyl, C(O)—O—C₂₋₃ alkenyl,
   O—R5, S—R5, N(H)R5,
   aryl, and
   (Y1)-aryl;
any mentioned C₁₋₆ alkyl and any mentioned C₂₋₃ alkenyl, if the C₂₋₃ alkenyl is not expressly labelled as being unsubstituted, is, independently from any other C₁₋₆ alkyl and C₂₋₃ alkenyl, unsubstituted or substituted by 1, 2, 3, 4, 5, or 6 identical or different substituents selected from a group consisting of oxo, OH, F, Cl, Br, I, NO₂,
   O—C₁₋₄ alkyl, O-(unsubstituted C₂₋₃ alkenyl),
   C(O)H, C(O)—C₁₋₄ alkyl, C(O)-(unsubstituted C₂₋₃ alkenyl),
   COOH, C(O)—O—C₁₋₄ alkyl, C(O)—O-(unsubstituted C₂₋₃ alkenyl),
   O—R5, S—R5, and N(H)R5.

In an embodiment,
the compounds of formulas (D6), (D6-F), (D5), and (D5-F) and the compounds of formulas (D6-I), (D6-F-I), (D5-I) and (D5-F-I) are in an alpha position to the keto group not substituted by an exocyclic C(O), said alpha position to the keto group is the C² and the C⁶ in formulas (D6), (D6-F) and in formulas (D6-I) and (D6-F-I), and the C² and the C⁵ in formulas (D5), (D5-F) and in formulas (D5-I) and (D5-I-F), respectively;
and/or
the C⁶ in formula (D6) (D6-F) and in formula (D6-I) and (D6-I-F) and the C⁵ in formula (D5), (D5-F) and in formula (D5-I) and (D5-I-F) is not substituted by oxo;
and/or
the compounds of formulas (D6), (D6-F), (D5), and (D5-F) and the compounds of formulas (D6-I), (D6-F-I), (D5-I) and (D5-F-I) are not substituted by oxo in beta position to the keto group, said beta position can be an endocyclic beta position or it can be an exocyclic beta position in case of respective substitution;
preferably,
the compounds of formulas (D6), (D6-F), (D5) and (D5-F) and the compounds of formulas (D6-I), (D6-F-I), (D5-I) and (D5-F-I) are not substituted by oxo in alpha and/or in beta position to the keto group, said beta position can be an endocyclic beta position or it can be an exocyclic beta position in case of respective substitution, and/or
R100, R101 and R102 are not oxo.

In an embodiment,
when any RINGALIPH is a cyclohexane, cyclopentane or cyclobutane and is substituted by an oxo residue, said oxo residue forms together with the C atom an endocyclic C(O) residue, then
RINGALIPH is not substituted by oxo in alpha position of said endocyclic C(O) residue, and/or
RINGALIPH is not substituted by oxo in the beta position of said endocyclic C(O) residue, said beta position can be an endocyclic beta position or it can be an exocyclic beta position in case of respective substitution;
preferably,
when any RINGALIPH is a cyclohexane, cyclopentane or cyclobutane then RINGALIPH is not substituted by oxo;
more preferably,
RINGALIPH is not substituted by oxo.

In an embodiment,
when in substrate a) any one of R1 and R2 is said aryl or is said C₂₋₃₀ alkenyl, wherein said C₂₋₃₀ alkenyl is substituted with aryl and said C₂₋₃₀ alkenyl together with its substituting aryl represents a vinylogous aryl,
then any Y2 in said aryl and in said vinylogous aryl is not an endocyclic unsaturated N and is not an endocyclic saturated unsubstituted N.

In an embodiment,
in formula (D6), formula (D6-F), formula (D6-I), and formula (D6-F-I) the three endocyclic bonds C³-C⁴, C⁴-C⁵ and C⁵-C⁶ are single bonds, and
in formula (D5), formula (D5-F), formula (D5-I) and formula (D5-F-I) the two endocyclic bonds C³-C⁴ and C⁴-C⁵ are single bonds.

In an embodiment,
any Y2 in any aryl and in any terminal aryl is not an endocyclic unsaturated N and is not unsubstituted saturated N, and/or
any Y2 in any RINGALIPH does is not an endocyclic unsaturated N and is not unsubstituted saturated N, and/or
any Y2 is not unsaturated N and is not unsubstituted saturated N.

Non-limiting examples for substrate a) are

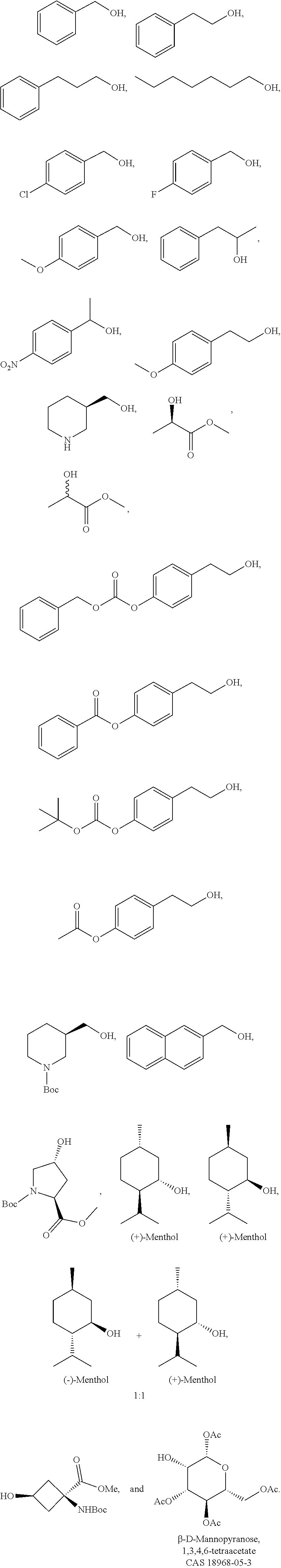

In an embodiment, substrate a) is compound of formula (500)

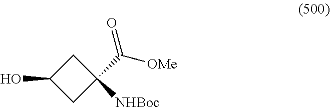 [SMILES: COC(=O)[C@]1(NC(=O)OC(C)(C)C)C[C@@H](O)C1]

In an embodiment, substrate a) is compound of formula (501)

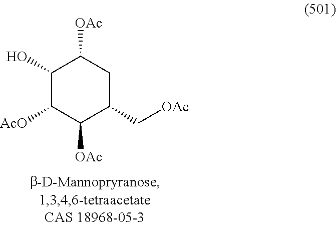 [SMILES: CC(=O)OC[C@H]1C[C@@H](OC(C)=O)[C@@H](O)[C@@H](OC(C)=O)[C@@H]1OC(C)=O]

Non-limiting examples for substrate b) are

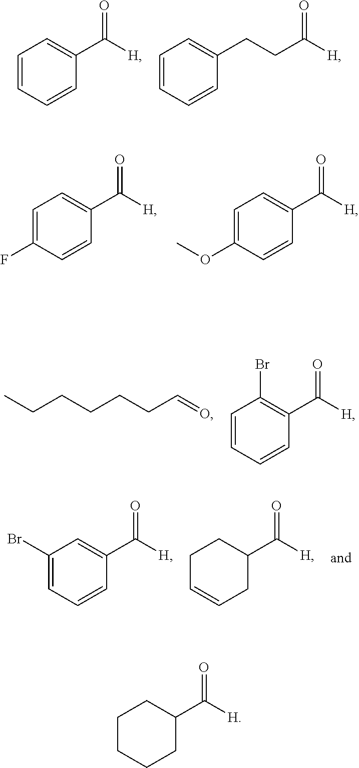

Non-limiting examples for substrate c) are

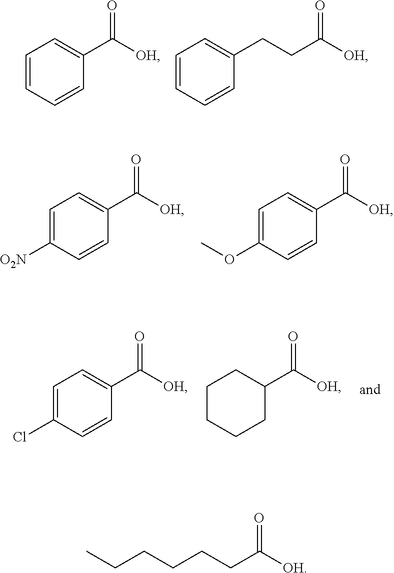

Non-limiting examples for substrate d) are

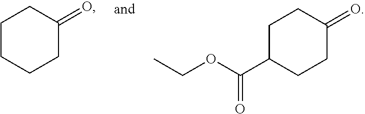

### Abbreviations and Definitions Used Throughout the Specification

ac acetyl
aq. aqueous
Barg bar gauge, pressure above atmospheric pressure pₐₜₘ, pₐₜₘ is approximately 1.013 bar
BPR back pressure regulation device
Boc tert-Butyloxycarbonyl
Bz Benzoyl
Cbz Benzyloxycarbonyl, also abbreviated with Z
CbzCl Benzyl chloroformate, also abbreviated with Z-chloride
CompEx comparative example
conv conversion
CV column volume(s)
DAST diethylaminosulfur trifluoride
DBU 1,8-Diazabicyclo[5.4.0]undec-7-ene
DCM Dicholoromethane
DIPEA N,N-diisopropylethylamine
EA ethyl acetate
EtOAc ethyl acetate
equiv molar equivalent
Ex example
FEP fluoroethylene-propylene
FFKM perfluoroelastomeric compound
FID flame ionization detector
LPR line pressure regulator
M molar
MFC mass flow controller
MIXDEV mixing device
MTBE Methyl tert-butyl ether
na not available
NPT National Pipe Taper
PE petroleum spirit 40-60
PEEK polyether ether ketone
PFA perflouralkoxy
PP polypropylene
Prod product
PTFE polytetrafluoroethylene
PVDF polyvinylfluorid
RESDEV residence device
RT room temperature
SEPDEV separation device
sat. saturated
sel selectivity
SS stainless steel
t_{Mix} mixing time
t_{Res} residence time
t_{Sep} separation time
TLC Thin Layer Chromatography
\dot{V}_{g} gaseous flow rate
\dot{V}ₗ liquid flow rate
Z Benzyloxycarbonyl, also abbreviated with Cbz

if not explicitly stated otherwise in the text.

### EXAMPLES

### Materials

All materials were obtained from commercial suppliers (TCI, Sigma Aldrich, Alfa Aesar, Acros Organics, ThermoScientific or VWR) and used without further purification unless otherwise noted. Ethyl acetate extra dry (99.900, water <50 ppm, AcroSeal®) was used as a solvent. Triethylamine 990% was acquired from ThermoScientific and trifluorotoluene 9800 from TCI was used.

Table 15 shows the sources of the substrates.

TABLE 15

| Substrate | Source |
|---|---|
| 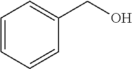 | Sigma Aldrich, 99+%, A.C.S. Reagent |
| 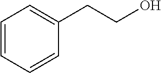 | Acros Organics, 99% |
| 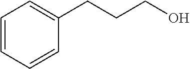 | Sigma Aldrich, 98% |
| 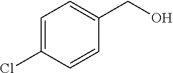 | Sigma Aldrich, 99% |
| 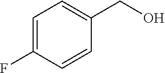 | Sigma Aldrich, 97% |
| 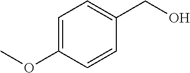 | Acros Organics, 98% |
| 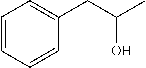 | TCI, 98+% |
| 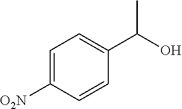 | Fluorochem, 99% |
| 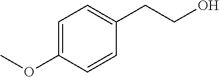 | Sigma Aldrich, 99% |
| 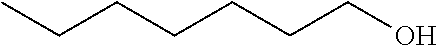 | Sigma Aldrich, 98% |
| 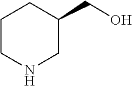 | Sigma Aldrich, 96% |
| 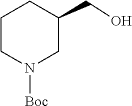 [SMILES: CC(C)(C)OC(=O)N1CCC[C@@H](CO)C1] | TCI, 95% |
| 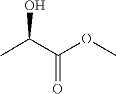 | Sigma Aldrich, 98%, 97% ee/GLC |
| 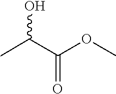 | TCI, 98%, racemic mixture |
| 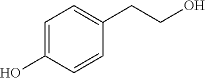 | Sigma Aldrich, 98% |
| 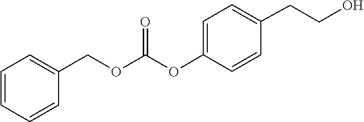 [SMILES: O=C(OCc1ccccc1)Oc1ccc(CCO)cc1] | Prepared according to Example 2 |
| 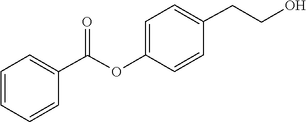 [SMILES: O=C(Oc1ccc(CCO)cc1)c1ccccc1] | Prepared according to Example 1 |
| 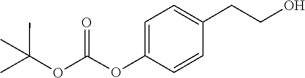 [SMILES: CC(C)(C)OC(=O)Oc1ccc(CCO)cc1] | Prepared according to Example 4 |
| 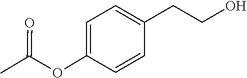 | Prepared according to Example 5 |
| 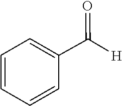 | Sigma Aldrich, 99% |
| 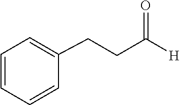 | Sigma Aldrich, 99% |
| 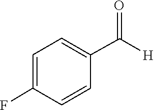 | Sigma Aldrich, 98% |
| 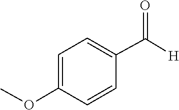 | Acros Organics, 99+% |
| 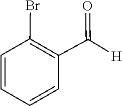 | Sigma Aldrich, 98% |
| 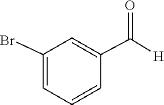 | Sigma Aldrich, 97% |
| 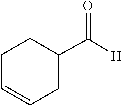 | Sigma Aldrich, 97% |
| 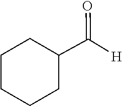 | Sigma Aldrich, 97% |
| 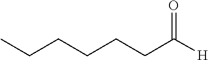 | Sigma Aldrich, 97% |
| 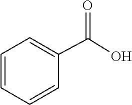 | Sigma Aldrich, >99% |
| 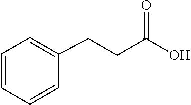 [SMILES: O=C(O)CCc1ccccc1] | Alfa Aesar, 99% |
| 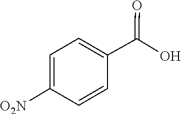 [SMILES: O=C(O)c1ccc([N+](=O)[O-])cc1] | Fluka, 99% |
| 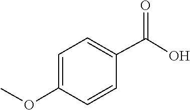 | Sigma Aldrich, 99% |
| 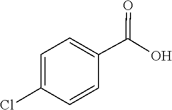 | Sigma Aldrich, 99% |
| 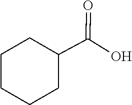 | Sigma Aldrich, 98% |
| 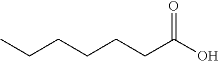 | Sigma Aldrich, >99% |
| 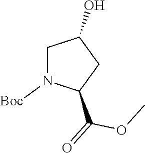 [SMILES: COC(=O)[C@@H]1C[C@@H](O)CN1C(=O)OC(C)(C)C] | Sigma Aldrich, >97% |
| 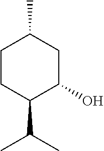 | TCI, >99% |
| (+)-Menthol | |
| 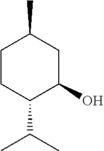 | TCI, >99% |
| (−)-Menthol | |
| 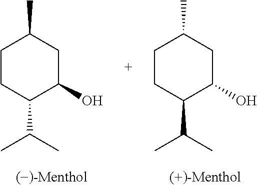 | TCI, >98% |
| 1:1 | |

### Method: NMR

### Offline NMR Analysis:

spectra were recorded on a NMR instrument of Bruker, Germany: ¹H were recorded at 500 MHz or 300 MHz, and ¹³C spectra at 126 MHz or 75 MHz, respectively, with a chemical shift relative to TMS expressed in parts per million (ppm). The samples were prepared in CDCl₃. The letters s, d, t and m are used to indicate singlet, doublet, triplet, and multiplet, respectively.

A Magritek Benchtop NMR (Spinsolve Ultra 43 MHz) was used for offline ¹⁹F NMR measurements of reaction mixtures. FEP NMR liners were inserted into standard NMR tubes to prevent corrosion.

Any so called yield ¹⁹F was determined offline using trifluorotoluene as internal standard, the respective signal is at −63.7 ppm. To calculate this yield ¹⁹F, the area of the NMR signal of the internal standard was compared to the area of the NMR signal of product or impurity, as the case may be, and adjusted to the used amounts.

### Inline NMR Analysis:

Inline NMR analysis was accomplished by recording ¹H and ¹⁹F spectra using a low field benchtop 43.795 MHz NMR spectrometer (Magritek GmbH, Germany, Spinsolve Ultra 43 MHz). Shims were performed with the PFA flow cell filled with ethyl acetate and referenced to the water peak at 2.10 ppm. Typically, a “QUICKSHIM: ALL” was performed in the Spinsolve software (Magritek) and shim values were below 0.4 Hz linewidth at 50%, below 7.5 Hz linewidth at 0.55% and a signal to noise ratio above 20′000.

The spectra were typically recorded in the reaction monitoring mode. ¹H NMR and ¹⁹F NMR were alternated with a wait time of 2 sec. ¹H NMR spectra were measured with a pulse angle of 90°, acquisition time of 6.4 s, repetition time of 7 s and a single scan. ¹⁹F NMR spectra were measured with a pulse angle of 90°, acquisition time of 0.82 s, repetition time of 3 s, a center frequency of −71, a bandwidth of 492 and 8 scans. The recorded spectra were automatically read and processed with an indirect hard model developed in PEAXACT® and ProcessLink®, S-PACT GmbH, Germany.

### Method: GC Analysis

GC analysis was performed on a Shimadzu GC FID 230 with a flame ionization detector (FID), using an RTX-5MS Cap. column (30 m×0.25 mm ID×0.25 μm) and helium as carrier gas (40 cm/sec⁻¹ linear velocity). The injector temperature was set to 280° C. After 1 min at 50° C., the temperature was increased by 25° C./min to 300° C. and kept constant at 300° C. for 4 min. FID was used for detection, the detector gases used for flame ionization were hydrogen and synthetic air (5.0 quality).

### Quantification by GC:

Selectivity GC in % refers to area % of the respective peak (selectivity of product or of by-product, as the case may be), the area % are based on the total area of all peaks of substrate, product and any by-products.
Conversion GC and yield GC in %: Quantification was done using trifluorotoluene as internal standard. To calculate the percentage, the area ratio between internal standard and substrate (conversion GC) or product or impurities (yield GC), as the case may be, was compared before and after the reaction.

### Method: Chiral GC Analysis

Chiral GC analysis was performed on an Agilent 7890A GC system equipped with a flame ionization detector (FID), using an Rt-BDEXse column (30 m×0.32 mm ID×0.25 μm df) (Restek GmbH, GER) and helium as carrier gas (0.5 mL min⁻¹ linear velocity). The injector temperature was set to 230° C. After 1 min at 60° C., the temperature was increased by 5° C./min to 180° C. and kept constant at 180° C. for 2 min. FID was used for detection, the detector gases used for flame ionization were hydrogen and synthetic air (5.0 quality). Stereoselectivity refers to area % of the respective peaks, the area % are based on the total area of all peaks of substrate, product and any by-products.

### Method: GC-MS Analysis

GC-MS analysis was performed using a Shimadzu GCMS-QP2010 SE, using an RTX-5MS column (30 m×0.25 mm×0.25 μm) and helium as carrier gas (40 cm/sec linear velocity). The injector temperature was set to 280° C. After 1 min at 50° C., the oven temperature was increased by 25° C./min to 300° C. and then kept at 300° C. for 3 min. The mass detector was a quadrupole with pre rods and electron impact ionization. The following settings were used in the detector: ion source temperature 200° C., interface temperature 310° C., solvent cut time 2 min 30 sec, acquisition mode scan, mass range m/z=50 till m/z=400.

### Method: HPLC Analysis

High performance liquid chromatography (HPLC) analysis was carried out on a C18 reversed-phase analytical column (150×4.6 mm, particle size 5 m) at 37° C. by using mobile phases A [water/acetonitrile 90:10 (v/v)+0.1 vol-% TFA] and B (acetonitrile+0.1 vol-% TFA, the vol % based on combined volume water and acetonitrile) at a flow rate of 1.5 mL/min. The follow gradient was applied: linear increase from 30% solution B to 100% B in 10 min, hold at 100% solution B for 3 min. All samples were prepared in HPLC grade acetonitrile and analyzed at 215 nm. For quantitative analysis a calibration was performed with phenylethanol, 2-fluoroethyl benzene, and trifluorotoluene.

In the examples it is noted where instead of the GC results selectivity GC, conversion GC and yield GC the respective results from HPLC analysis are given.

### Method: Flash Column Chromatography

Automated flash column chromatography was performed on a Biotage Isolera system using columns packed with KP-SIL, 60 Å (32 to 63 m particle size) silica. Eluent was petroleum spirit 40-60 with an increasing gradient of ethyl acetate.

### Method: TLC

Analytical thin-layer chromatography (TLC) was carried out using Merck silica gel 60 GF254 plates. Compounds were visualized by means of UV or by using KMnO₄ or a ninhydrine solution (400 mg ninhydrine in a total volume of 200 mL n-butanol with 0.5 vol % acetic acid and 4.5 vol % water, the vol % begin based on the total volume). Eluent was the same as used for Flash Column Chromatography or it is stated.

### Material of Construction (MoC)

SF₄ is in general not very corrosive but as SF₄ gets in contact with water, even in form of moisture, HF is immediately formed. So, a similar material of construction (MoC) as when working with HF has to be used. Therefore, wetted parts have to be made out of PEEK, PTFE, PFA, PP, FEP, stainless steel or FFKM. Glass-made material can not be used. Additionally, it is highly recommended to check all the material for corrosion regularly and perform daily leak tests before starting the system. To prevent corrosion it has to be ensured that the system is flushed by N₂ 6.0 before starting the SF₄ line to remove traces of moisture in the system, which could lead to HF formation. In this way a longer lifetime of the instruments can be achieved.

### (A) Flow Configuration

The flow configuration is shown in FIG. 1 and described in detail as follows:

Standard PFA tubing (0.8 mm or 1.6 mm i.d.), PTFE fittings and T-pieces were used in the flow setups. For the gaseous feed stainless steel fittings from Fitok Group, US, were used. For pumping reagent solutions, syringe pumps (Syrris Asia, UK) equipped with 1 mL and 0.5 mL syringes were used. All pumps were used with check valves (Upchurch, CV-3321, IDEX Corporation, US) and internal pressure sensors. The pressure limit of the pumps was set to 4 bar to prevent pushing the liquid back to the mass flow controller MFC for the gaseous feed. Above 4 bar pressure, the pumps would turn-off automatically for safety reasons. Before using the pumping systems, they were calibrated by pumping for a specified time and checking the mass balance. All pumps were found to dose within +3%. For optimization studies and small-scale experiments, a 6-port valve with a 5 mL sample loop having an inject position and a load position was used. The solvent was either pumped directly through a check valve (Upchurch, CV-3321, IDEX Corporation, US), to prevent backflow of the liquid phase, into a MIXDEV, a T-piece, this happened in the load position of the sample loop, or first through the 5 mL sample loop and then through the check valve into the T-piece for mixing the liquid feed with the gas feed, this happened in the inject position of the sample loop.

For the gaseous feed, a bottle of SF₄ (1 kg, 99%, 9.65 bar pressure, lot 1377769, abcr GmbH, Germany) was connected to a magnetic valve (Type 0330, PVDF, NPT 4, FFKM, Burkert Werke GmbH, Germany) with a manual power switch outside the fume hood. This valve was connected to a 3-way valve (stainless steel), which was connected to a bottle of N₂ (6.0, air liquide) to flush the whole system. After the 3-way valve, a line pressure regulator LPR (KPR1FJC412A20000, Swagelok Company, US) was used to reduce the pressure to 4 bar. The flow was controlled by a mass flow controller MFC (LOW-ΔP-FLOW F-201DV, Bronkhortst High-Tech B.V., NL) calibrated at p₁=10 barg, p₂=5 barg, T=30° C. (accuracy ±1%) connected via RS-232 to a controller. Flow rates of the mass flow controller MFC are given in mLₙ/min, where n presents measurement under standard conditions (Tₙ=0° C., pₙ=1.013 bar). After the MFC, a check valve (wetted material: SS, Kalrez®, Fitok Group, US) was used to prevent backflow of the liquid phase. After this check valve the gas flow was mixed with the solvent flow with the MIXDEV, the T-piece (Tee Body IDEX H&S P-712-01, PEEK, 0.020″ (0.50 mm) thru hole), whose outlet was connect to a RESDEV, a reactor coil. The reactor coil (0.8 mm i.d., 4.6 mL volume) was placed into a heated water bath on a magnetic stirrer. After the reactor coil, the flow then passed through an inline NMR analysis (as described above) and the outlet tubing was connected to an adjustable back pressure regulator BPR (20 bar max, Zaiput Flow Technologies, US) pressurized by compressed air to 2.5 barg.

The reaction mixture subsequently passed into a SEPDEV, a membrane separator (SEP-10, Zaiput Flow Technologies, US) using a hydrophobic membrane (Whatman 7585-004, PTFE Membrane, WTP Range, 0.5 μm pore size, 47 mm circle (100 pcs), Whatman plc, UK) cut to the required size of the membrane separator, to separate the reaction mixture into a hydrophilic stream, which was retained by the membrane and was a gaseous stream and which contained any residual amount of SF₄, and a hydrophobic stream, which passed through the membrane and was a liquid stream and which contained the solvent and any dissolved compounds.

The gaseous stream was diluted via a T-piece with water (300 μL/min) and then collected in a stirred first quench bottle containing aqueous NaOH (5 wt %) and a few drops of phenolphthalein as a pH indicator.

The liquid stream was forwarded to a 4-way valve (4-way valve PEEK bulkhead single “T” Flow, IDEX Corporation, US). One outlet was a short tubing for sampling. Another outlet was connected via a T-piece to dilute the liquid stream with water (200 μL/min) into a stirred second quench bottle containing aqueous NaOH (5 wt %) and a few drops of phenolphthalein as a pH indicator. The headspace of that second quench bottle was connected into the first quench bottle from the gaseous stream. The headspace of this first quench bottle was connected to a third quench bottle containing aqueous NaOH (10 wt %) and a few drops of phenolphthalein as a pH indicator. The head space of this third quench bottle was connected to a tubing going to the back of the fume cupboard (not shown in FIG. 1).

The dilution of both of the gaseous stream and the liquid stream with water served the purpose to prevent NaF formation in the tubing, which can lead to clogging. The water was dosed via respective pumps, which are not shown in FIG. 1. Additionally, the quench bottles were stirred by magnetic stirrers and if necessary cooled with an ice bath.

(B) Fluorination Procedure Exemplified with Phenylethanol

Phenylethanol was reacted with SF₄ as described in this (B) Fluorination Procedure and as shown in Scheme 6, the (B) Fluorination Procedure was done with the (A) Flow Configuration.

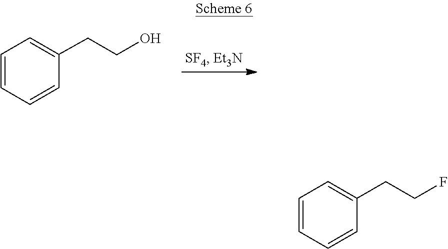 [SMILES: OCCc1ccccc1 ; FCCc1ccccc1]

The feed solution FEED1 containing the substrate was prepared in a volumetric flask. Phenylethanol (305 mg, 2.5 mmol), triethylamine (254 mg, 2.5 mmol, 1 equiv) and trifluorotoluene (0.178 mg, 1.25 mmol) were dissolved in ethyl acetate (5 mL, resulting in a concentration of 0.5 M of phenylethanol and 0.25 M of trifluorotoluene). After mixing, the flask was sealed under argon atmosphere, and the solution was taken out using a syringe and a needle and then injected into the sample loop in the load position.

In order to remove moisture from the system before running the reaction, the system was flushed with N₂ (15 mLₙ/min) for 20 min, and then, the EtOAc pump was started (1.00 mL/min) and the whole system was flushed for 15 min. The heating bath for the reactor coil (4.6 mL internal volume) was heated to and kept at 50° C. After reaching in the heating bath the temperature of 50° C., the 3-way valve was turned and SF₄ feed FEED2 was started. The gaseous flow rate \dot{V}_{g} was set to 25 mLₙ/min and the mixing with EtOAc (liquid flow rate \dot{V}ₗ of 1 mL/min) continued for 4 min. Then, the gaseous flow rate \dot{V}_{g} was reduced to 12 mLₙ/min (1 equiv SF₄) and the system was left to equilibrate until all gas was dissolved after the T-piece mixer (no N₂ traces left). The 6-way valve was then turned to the inject position and the substrate/base loaded sample loop was fed into the reactor coil (t_{Res} 276 sec). When steady state was observed using the in-line NMR analysis, 30 sec were waited until sampling into the collection flasks was started to ensure that the reaction mixture had passed through the remaining volume of the system and had reached the sampling outlet. For sampling, the 4-way valve was turned and the outlet tubing was placed into 4 mL vials containing quench solution and a stirring bar. The following order of samples was collected, each sample was collected for 30 sec if not otherwise stated:
1) NaOH 5%
2) aq. sat. solution of NaHCO₃
3) sampling into an FEP NMR liner without a quench
4) NaOH 5%: sampling for 2 min 30 sec
5) NaOH 5%

The sample 3) in the FEP NMR liner was analyzed by offline NMR analysis (¹H and ¹⁹F). From the quenched samples 1), 2), 4) and 5) the organic phase was taken (10 μL) and diluted in MeCN (990 μL) for GC analysis, the mean value of the 4 GC values (while eliminating any obvious outlier) of these 4 samples is reported under GC results herein. The same samples were analyzed by GC-MS using their molecular weights for identification. After finishing the fluorination experiment, the system was flushed with N₂ (15 mLₙ/min) and EtOAc (1 mLₙ/min) for 10 min. Then, the solvent pump was turned off and the system was flushed with N₂ (15 mLₙ/min) for 10 more min.

### (C) Isolation Procedure

The following isolation procedure after the (B) Fluorination procedure was used: the organic phase of sample 4) was separated from the aqueous phase and washed with an aq. sat. solution of NaHCO₃ twice and with an aq. sat. solution of NaCl once. The organic phase was dried over Na₂SO₄ and filtered. The organic solvent was evaporated and dried at 2 mbar at 40° C. to afford the desired fluorinated products. If the boiling points of the products were too close to ethyl acetate (bp=77° C.), fractional distillation was carried out. For the products where purity was not satisfying, flash column chromatography was performed for further purification. Isolated compounds were characterized by NMR analysis. Isolated yield takes into account the purity.

### Example 1—O-Benzoyl Tyrosol

The synthesis of Bz-protected Tyrosol was conducted based on a procedure published by Liang et al, J. Chin. Pharm. Sci. 2014, 23, 446-453, point 4.2 Synthesis of 2(4-benzoyloxyphenyl)ethanol (la), Route A, as shown in Scheme 1:

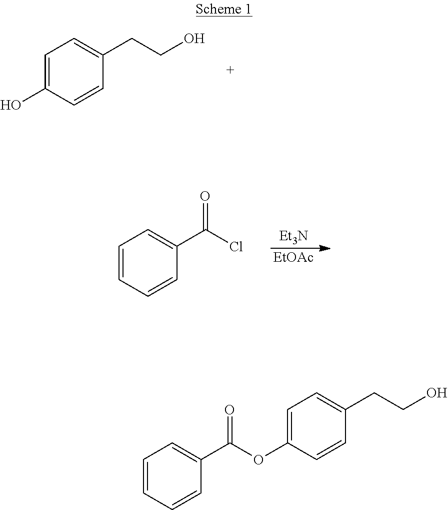 [SMILES: "EtOAc" ; "Et3N" ; OCCc1ccc(O)cc1 ; O=C(Cl)c1ccccc1 ; O=C(Oc1ccc(CCO)cc1)c1ccccc1]

5.09 g of Tyrosol (0.037 mol, 1 equiv) was dissolved in ethyl acetate (50 mL) and mixed with triethylamine (5 mL). The mixture was then cooled with an ice bath, and benzoyl chloride (4.55 mL, 1.06 equiv) dissolved in ethyl acetate (10 mL) was added. The ice bath was removed and the reaction was allowed to proceed for 1 h while the temperature increased to room temperature. The completion of the reaction was verified using TLC (PE:EA was 1:1 v/v). The reaction mixture at the end of the reaction was a solution containing precipitated triethylamine hydrochloride salt, the reaction mixture was filtered to remove the triethylamine hydrochloride salt and washed three times with an aq. sat. solution of NaHCO₃ and with an aq. sat. solution of NaCl once. The washed solution was dried with anhydrous Na₂SO₄ and the volume was reduced with a rotary evaporator (40° C. and 10 mbar) to approximately 5 mL. Then, petrolether (12 mL) and chloroform (3 mL) were added and a homogenous solution was obtained after heating to 50° C. After cooling to 25° C. the product crystallized. The crystallized product was isolated by filtration and dried in vacuum oven overnight (2 mbar, 50° C.). This procedure gave a selectivity GC of 80%, which was further increased by flash column chromatography using a linear gradient from 13 to 100% (v/v) EA over 10 CV. A white solid (1.97 g, 8.14 mmol, selectivity GC of 95%) was obtained giving 22% isolated yield. ¹H and ¹³C NMR analysis confirmed the structure.

### Example 2—O-Benzyloxycarbonyl Tyrosol

The synthesis of Cbz protected Tyrosol was conducted based on a procedure published by Liang et al, J. Chin. Pharm. Sci. 2014, 23, 446-453, point 4.2 Synthesis of 2(4-benzoyloxycarbonylphenyl)ethanol (12a), Route A, as shown in Scheme 2:

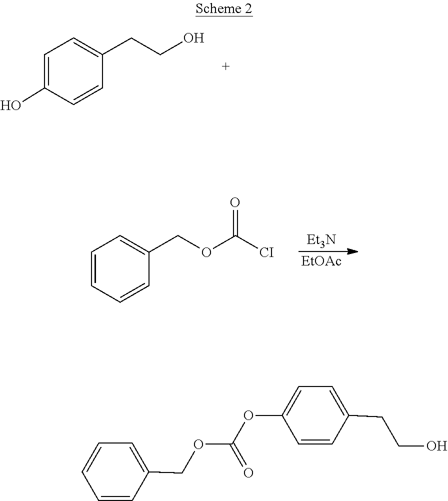

3.02 g of Tyrosol (0.022 mol, 1 equiv) was dissolved in ethyl acetate (30 mL) and mixed with triethylamine (3 mL). The mixture was then cooled in an ice bath, and benzyl chloroformate (CbzCl) (3.99 g, 1.06 equiv) dissolved in ethyl acetate (6 mL) was added. The ice bath was removed and the reaction was allowed to proceed for 1 h 40 min while the temperature increase to room temperature. The completion of the reaction was verified using TLC (PE EA was 1:1 v/v). The reaction mixture at the end of the reaction was a solution containing precipitated triethylamine hydrochloride salt, the reaction mixture was filtered to remove the triethylamine hydrochloride salt and washed three times with an aq. sat. solution of NaHCO₃ and with an aq. sat. solution of NaCl once. The washed solution was dried with anhydrous Na₂SO₄ and the solvent was evaporated with a rotary evaporator (40° C. and 10 mbar) to dryness under reduced pressure of 5 mbar. Purification was achieved by flash column chromatography using a linear gradient from 10 to 80% (v/v) EA over 10 CV. A colorless oil (4.68 g, 17.2 mmol, selectivity GC of 92%) was obtained giving 78% isolated yield. ¹H and ¹³C NMR confirmed the structure.

### Example 4—O-tert-Butyloxycarbonyl Tyrosol

The synthesis of Boc protected Tyrosol was conducted based on a procedure published by Saito et al, Tetrahedron Lett. 2010, 51, 6915-6917, point 2 Typical experimental procedure, Compound (1h) in Table 2, as shown in Scheme 4:

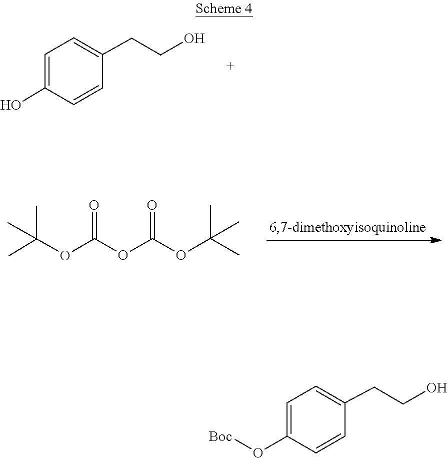 [SMILES: OCCc1ccc(O)cc1 ; CC(C)(C)OC(=O)OC(=O)OC(C)(C)C ; CC(C)(C)OC(=O)Oc1ccc(CCO)cc1]

Tyrosol (1.38 g, 10 mmol), di-tert-butyldicarbonate (2.62 g, 1.10 equiv) and 6,7-dimethoxyisoquinoline (19.4 mg, 5 mol %) were placed in a flask equipped with a stirring bar under Argon atmosphere. Dry dichloromethane (15 mL) was added and the heterogenous mixture was stirred at RT overnight to provide a clear homogenous solution, which was diluted with ethyl acetate (35 mL). Then, the mixture was washed with aq. HCl (1 M, 2 times 10 mL), aq. sat. solution of NaCl (15 mL), aq. NaOH (5%, 2 times 10 mL), and aq. sat. solution of NaCl (15 mL). Then the organic phase was dried over Na₂SO₄ and after filtration the volatiles were evaporated with a rotary evaporator at 40° C. and 5 mbar. To separate the main by-products flash column chromatography was performed, using a linear gradient from 5 to 80% (v/v) EA over 15 CV. After evaporating the solvents with a rotary evaporator at 40° C. and 5 mbar a white solid (1.19 g, 5.01 mmol, selectivity GC of 94%) was obtained with 50% isolated yield. ¹H and ¹³C NMR analysis confirmed the structure.

### Example 5—Acetic Acid Ester of Tyrosol

The synthesis of Ac protected Tyrosol was conducted based on a procedure published by Liao et al, Org. Lett. 2022, 24, 4207-4211, compound (4b) in scheme 4, general procedure III: Site-Selective acylation of phenol derivatives (see ESI). A reaction using acetic acid anhydride is not reported, however the conditions were applied as shown in Scheme 5:

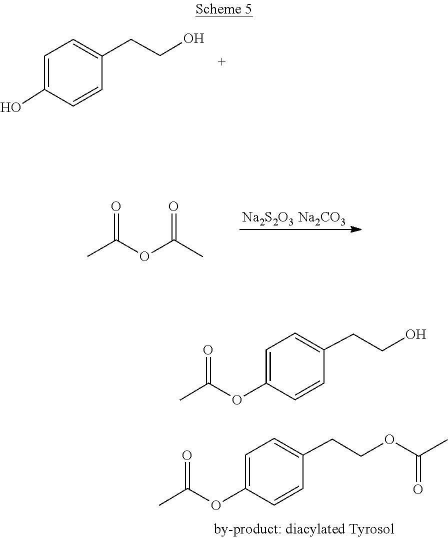

Tyrosol (2.07 g, 15 mmol), acetic anhydride (1.53 g, 1 equiv), anhydrous sodium thiosulfate (0.452 g, 0.2 equiv) and sodium carbonate (1.59 g, 1 equiv) were mixed in dry dimethyl formamide (50 mL) under Argon atmosphere. The heterogenous mixture was stirred at 70° C. overnight. Then, 20 mL diethyl ether and 20 mL water were added and phases were separated. The aqueous phase was washed with diethyl ether (4 times 20 mL). The combined organic layers were washed with aq. sat. solution of NaCl (2 times 15 mL) and dried over Na₂SO₄. After filtration and evaporation of the volatiles with a rotary evaporator at 40° C. and 5 mbar a colorless oil (1.14 g) was obtained. Purity GC was 71%. To separate the main by-products flash column chromatography was performed, using dichloromethane with a linear gradient from 1 to 2100 (v/v) methanol over 17 CV. After evaporating the solvents with a rotary evaporator at 40° C. and 5 mbar a colorless oil (825 mg, 4.58 mmol, selectivity GC of 77%) was obtained. The main by-product was the diacylated Tyrosol. ¹H and ¹³C NMR analysis confirmed the structures.

### Examples 10 to 30—Substrate Phenylethanol

Phenylethanol was reacted according to (B) Fluorination Procedure; deviations from the protocol of the (B) Fluorination Procedure are given in Table 1, the row (B) gives the respective values for (B) Fluorination Procedure.

TABLE 1

| | | | | liquid | gaseous | | | | | |
|---|---|---|---|---|---|---|---|---|---|---|
| | | | | Flow | Flow | | | | | |
| | | | Coil | Rate | Rate | | | conv | yield | sel |
| | | T | Vol | \dot{V}ₗ | \dot{V}_{g} | SF₄ | t_{Res} | GC | GC | GC |
| Ex | Solvent | [° C.] | [mL] | [mL/min] | [mLₙ/min] | [equiv] | [sec] | [%] | [%] | [%] |
| (B) | EtOAc | 50 | 4.6 | 1 | 12 | 1 | 276 | >99 | 84 | 85 |
| 12 | EtOAc | 25 | 1.6 | 0.5 | 6.1 | 1 | 192 | 95 | 72 | 73 |
| 15 | EtOAc | 50 | 1.6 | 0.5 | 6.1 | 1 | 192 | 97 | 83 | 89 |
| 21 | EtOAc | 20 | 1.6 | 1 | 12.1 | 1 | 96 | 98 | 77 | 79 |
| (2)(3) | | | | | | | | | | |
| 22 | EtOAc | 20 | 1.6 | 1 | 12.1 | 1 | 96 | 98 | 73 | 74 |
| (2)(3) | | | | | | | | | | |
| 23 | EtOAc | 20 | 1.6 | 1 | 12.1 | 1 | 96 | 98 | 78 | 80 |
| (2)(3) | | | | | | | | | | |
| 24 | EtOAc | 50 | 4.6 | 1 | 24.2 | 1 | 276 | >99 | 84 | 87 |
| (4) | | | | | | | | | | |
| 25 | EtOAc | 50 | 4.6 | 0.5 | 12.1 | 1 | 552 | >99 | 78 | 81 |
| (4) | | | | | | | | | | |
| 26 | EtOAc | 50 | 4.6 | 1 | 36.3 | 1 | 276 | 98 | 71 | 74 |
| (4) | | | | | | | | | | |
| 27 | EtOAc | 50 | 10.8 | 0.63 | 7.7 | 1 | 1030 | 96 | 77 | 93 |
| 28 | EtOAc | 50 | 10.8 | 0.63 | 11.4 | 1.5 | 1030 | 96 | 79 | 93 |
| (5) | | | | | | | | | | |
| 29 | EtOAc | 75 | 10.8 | 0.63 | 7.7 | 1 | 1030 | >99 | 70 | 71 |
| 30 | EtOAc | 50 | 10.8 | 0.94 | 17.0 | 1.5 | 686 | 95 | 80 | 92 |

(2) instead of GC analysis HPLC analysis was done, the results selectivity, conversion and yield are from HPLC analysis
(3) Example 21 employed a static mixer and Example 22 employed PTFE beads for mixing
   Static mixer: PFA tubing (10 cm length, 3.2 mm i.d.) containing 3 mm o.d. polypropylene Kenics static mixers
   PTFE beads: PFA tubing (10 cm length, 1.6 mm i.d.) containing Poly(tetrafluoroethylene) powder with >40 μm particle size (Sigma Aldrich, US) (self-packed, fixed with glass wool on both sides)
   Example 23 employed the T-piece as described in (A) Flow Configuration.
   The static mixer and the PTFE beads were inserted between the T-piece and the coil.
(4) In Examples 24, 25 and 26 the substrate concentration was different from (B) Fluorination Procedure (0.5 M):
   Examples 24 and 25: 1 M substrate, 1 equiv SF₄
   Example 26: 1.5 M, 1 equiv SF₄
(5) In Examples 28 and 30 1.5 equiv SF₄ were used

### Examples 35 to 38—Substrate Phenylethanol—the Base

Phenylethanol was reacted according to (B) Fluorination Procedure; deviations from the protocol of the (B) Fluorination Procedure are given in Table 11, the row (B) gives the respective values for (B) Fluorination Procedure.

TABLE 11

| | | | conv | yield | sel |
|---|---|---|---|---|---|
| | | base | GC | GC | GC |
| Ex | base | [equiv] | [%] | [%] | [%] |
| (B) | Et₃N | 1 | >99 | 84 | 85 |
| CompEx 30 | — | — | 96 | 38 | 36 |
| 35 | Et₃N | 3 | 99 | 73 | 88 |
| 36 | DIPEA | 1 | >99 | 99 | 95 |
| 37 | Piperidine | 1 | 77 | 54 | 51 |
| 38 | Pyridine | 1 | 95 | 53 | 60 |

### Examples 16 to 20 and 31—Substrate Phenylethanol—Solvents

Phenylethanol was reacted according to (B) Fluorination Procedure; deviations from the protocol of the (B) Fluorination Procedure are given in Table 12, the row (B) gives the respective values for (B) Fluorination Procedure.

TABLE 12

| | | | liquid | gaseous | | | | | |
|---|---|---|---|---|---|---|---|---|---|
| | | | Flow | Flow | | | | | |
| | | Coil | Rate | Rate | | | conv | yield | sel |
| | | Vol | \dot{V}ₗ | \dot{V}_{g} | SF₄ | t_{Res} | GC | GC | GC |
| Ex | Solvent | [mL] | [mL/min] | [mLₙ/min] | [equiv] | [sec] | [%] | [%] | [%] |
| (B) | EtOAc | 4.6 | 1 | 12 | 1 | 276 | >99 | 84 | 85 |
| 16 | C₆H₁₂ | 4.6 | 1 | 12.1 | 1 | 42 | >99 | 93 | 98 |
| (2) | (1) | | | | | | | | |
| 17 | EtOAc | 4.6 | 1 | 12.1 | 1 | 276 | >99 | 93 | 95 |
| (2) | | | | | | | | | |
| 18 | Diisopropylether | 4.6 | 1 | 12.1 | 1 | 42 | >99 | 87 | 87 |
| (2) | (1) | | | | | | | | |
| 19 | Isopropyl | 4.6 | 1 | 12.1 | 1 | 276 | >99 | 89 | 91 |
| (2) | acetate | | | | | | | | |
| 20 | MTBE | 4.6 | 1 | 12.1 | 1 | 276 | >99 | 82 | 90 |
| (2) | | | | | | | | | |
| 31 | DCM | 4.6 | 1 | 12.1 | 1 | 276 | >99 | 85 | 92 |
| (3) | | | | | | | | | |

(1) in case of cyclohexane and Diisopropylether a biphasic gaseous-liquid-flow regime was observed in contrast to a mono-phasic liquid flow regime in case of EtOAc. The gas bubbles influence strongly the real residence time in the reactor coil based on a real gaseous flow rate \dot{V}_{g,real} of 5.3 mL/min (at 50° C., ca. 2.5 bar and a density of SF₄ of 0.0036 g/mL), so the real residence time of Examples 16 and 18 as stated in Table 12 is an estimation based on these values.
(2) instead of GC analysis HPLC analysis was done, the results selectivity, conversion and yield are from HPLC analysis
(3) Example 31 was done with 0.5 M substrate in dichloromethane (instead of 0.5 M in EtOAc)

### Examples 52 to 59—Substrate Phenylpropionic aldehyde

Phenylpropionic aldehyde was reacted as shown in Scheme 7 with SF₄ according to (B) Fluorination Procedure with phenylpropionic aldehyde as substrate instead of phenylethanol; deviations from the protocol of the (B) Fluorination Procedure are:
1 M aldehyde (instead of 0.5 M of phenylethanol in (B)), 1 M Et₃N (instead of 0.5 M of phenylethanol in (B)) to have 1 equiv of Et₃N
further deviations are given in Table 2.

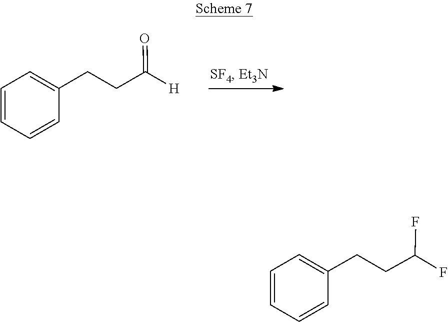 [SMILES: O=CCCc1ccccc1 ; FC(F)CCc1ccccc1]

TABLE 2

| | | Flow | Flow | | | | | | |
|---|---|---|---|---|---|---|---|---|---|
| | | Rate | Rate | | | | conv | yield | sel |
| | T | \dot{V}ₗ | \dot{V}_{g} | SF₄ | Base | t_{Res} | GC | GC | GC |
| Ex | [° C.] | [mL/min] | [mLₙ/min] | [equiv] | [equiv] | [sec] | [%] | [%] | [%] |
| 52 | 50 | 1 | 24.2 | 1 | 1 | 276 | 76 | 68 | 71 |
| 53 | 75 | 1 | 24.2 | 1 | 1 | 276 | 98 | 95 | 94 |
| 54 | 75 | 0.5 | 12.1 | 1 | 1 | 552 | 98 | 97 | 94 |
| 55 | 90 | 1 | 24.2 | 1 | 1 | 276 | 97 | 95 | 94 |
| 56 (a) | 50 | 1 | 36.3 | 3 | 3 | 648 | 78 | 77 | 73 |
| 57 (a) | 50 | 0.5 | 36.3 | 6 | 3 | 1296 | 82 | 80 | 76 |
| 58 (b) | 90 | 0.5 | 6.1 | 1 | 1 | 552 | 94 | 90 | 91 |
| 59 (b) | 90 | 0.5 | 9.2 | 1.5 | 1 | 552 | 95 | 92 | 92 |

(a) 10.8 mL coil instead of 4.6 mL coil, benzaldehyde instead of phenylpropionic aldehyde
(b) Examples 56 to 59 were done with 0.5M phenylpropionic aldehyde

### Examples 60 to 80—Substrate: Various Alcohols

The examples shown in Table 3 were done according to (B) Fluorination Procedure; the row (B) gives the respective values for (B) Fluorination Procedure.

The alcohols reacted with SF₄ as shown in Scheme A, the hydroxyl residue of the alcohol was exchanged against fluorine providing the respective (mono)fluorine compound.

TABLE 3

| | | conv | yield | isol. | sel |
|---|---|---|---|---|---|
| | | GC | GC | yield | GC |
| Ex | Substrate | [%] | [%] | [%] | [%] |
| 60A (m) | 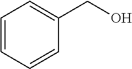 | 97 | 65 | na | 85 |
| 60B (m) | 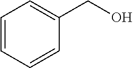 | 97 | 83 | na | 81 |
| (B) | 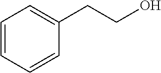 | >99 | 84 | 83 | 85 |
| 61 | 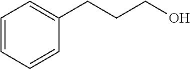 | >99 | 79 | 75 | 83 |
| 62 | 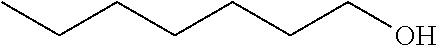 | >99 | 84 | na | 94 |
| 63 | 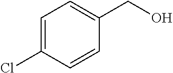 | 97 | 53 | na | 79 |
| 64 | 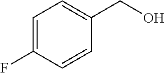 | 97 | 66 | na | 85 |
| 65 | 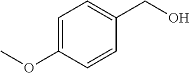 | >99 | 95 | na | 98 |
| 66A (d) | 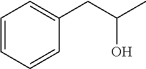 | 98 | 57 | na | 57 |
| 66B (d) | 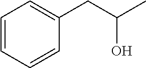 | 98 | 60 | na | 60 |
| 67 | 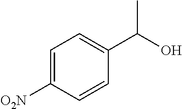 | 94 | 54 | na | 60 |
| 68 | 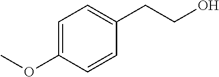 | >99 | 67 | na | 79 |
| 69 | 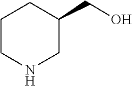 | na | 54 (c) | na | na |
| 70 (b) | 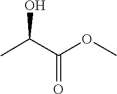 | >99 | 81 | na | 92 |
| Comp Ex 70 (e) | 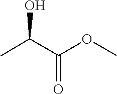 | >99 | 35 | na | 29 |
| 71 (a) | 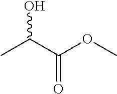 | >99 | 86 | na | 90 |
| 72 | 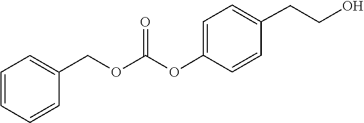 [SMILES: O=C(OCc1ccccc1)Oc1ccc(CCO)cc1] | 96 | 99 | 87 | 92 |
| 73 | 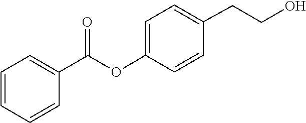 [SMILES: O=C(Oc1ccc(CCO)cc1)c1ccccc1] | 99 | 85 | 98 | 96 |
| 74 | 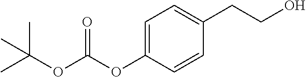 [SMILES: CC(C)(C)OC(=O)Oc1ccc(CCO)cc1] | 99 | 98 | 95 | 97 |
| Comp Ex 74 (f) | 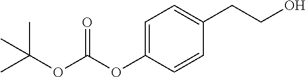 [SMILES: CC(C)(C)OC(=O)Oc1ccc(CCO)cc1] | >99 | 23 | na | 38 |
| 75 | 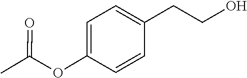 | 99 | 87 | 97 | 92 |
| 76 | 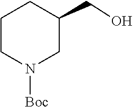 [SMILES: CC(C)(C)OC(=O)N1CCC[C@@H](CO)C1] | >99 | 81 | 74 | 87 |
| 77 | 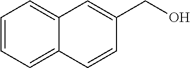 [SMILES: OCc1ccc2ccccc2c1] | 98 | 75 (g) | na | 88 |
| 78 | 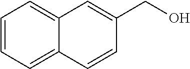 [SMILES: OCc1ccc2ccccc2c1] | 99 | 73 (h) | 82 | 90 |
| 79 (i) | 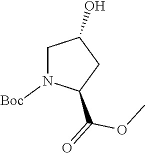 [SMILES: COC(=O)[C@@H]1C[C@@H](O)CN1C(=O)OC(C)(C)C] | 95 | 79 | 67 | 83 |
| 80a (j) | 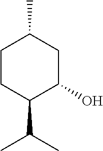 | 87 | 58 | na | 63 |
| | (+)-Menthol | | | | |
| 80b (j) | 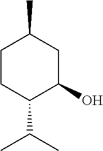 | 87 | 60 | na | 65 |
| | (−)-Menthol | | | | |
| 80c (j) | 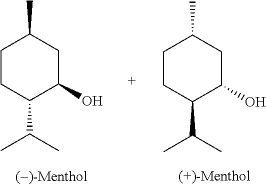 | 86 | 58 | na | 63 |
| | 1:1 | | | | |
| 80d (k) | 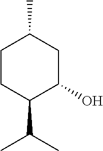 | 72 | 56 | na | 56 |
| | (+)-Menthol | | | | |
| 80e (k) | 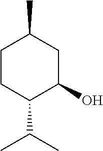 | 86 | 60 | na | 62 |
| | (−)-Menthol | | | | |
| 80f (l) | 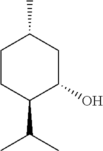 | 99 | 69 | na | 68 |
| | (+)-Menthol | | | | |
| 80g (I) | 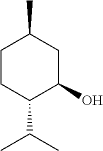 | 97 | 68 | na | 65 |
| | (-)-Menthol | | | | |
| 80h (1) | 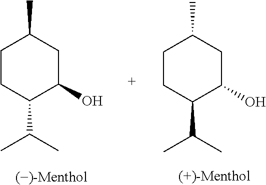 | 94 | 69 | na | 64 |
| | 1:1 | | | | |

(a) The substrate was racemic methyl lactate, a 1:1 mixture of (D)-methyl lactate and (L)-methyl lactate. The product was racemic as well as determined by Chiral GC Analysis. The Table heading ″Selectivity″, in this example, does not pertain to discrimination of the two possible enantiomers which could be obtained (i.e., if the reaction had or had not changed the configuration).
(b) The substrate was enantiopure (L)-methyl lactate. Chiral GC Analysis revealed a product stereoselectivity of 99%. The Table heading ″Selectivity″, in this example, does not pertain to discrimination of the two possible enantiomers which could be obtained (i.e., whether the reaction proceeded with inversion or retention of configuration).
(c) 2 equiv SF₄ (instead of 1 equiv SF₄), yield is yield ¹⁹F NMR (instead of GC yield)
(d) Example 66A was done with Et₃N, Example 66B with DIPEA as the base.
(e) Comparative Example 70: Example 70 was repeated with the sole difference that no base was added. Chiral GC Analysis revealed a stereoselectivity of only 72%. The Table heading ″Selectivity″, in this example, does not pertain to discrimination of the two possible enantiomers which could be obtained (i.e., whether the reaction proceeded with inversion or retention of configuration).
(f) Comparative Example 74 Example 70 4as repeated with the sole difference that no base was added. GC analysis showed many small peaks.
(g) Example 77 with 75 % yield GC was done with a concentration of substrate of 0.2M in dichloromethane, 1 equiv SF₄, 50° C., tRes 276 sec = 4 min 36 sec, which gives a reaction time of ca. 5 min (factor 1.1). Gustafsson reports a yield of 55% in Table 1 Entry 3 (0.2M in Dichloromethane, 1 equiv DAST, 70° C., reaction time 16 min).
(h) Example 78 with 73 % yield GC was done with a concentration of substrate of 0.5M in EtOAc, 1 equiv SF₄, 50° C., t_{Res} 276 sec = 4 min 36 sec, which gives a reaction time of ca. 5 min (factor 1.1).
(i) Tamboli discloses nosyl fluoride (13) as a deoxyfluorinating agent for N-Boc-4-hydroxy-L-proline methyl ester (2), reported yield is 52%. Tamboli also discloses DAST as deoxyfluorinating agent, reported yield is 66%. The Table heading ″Selectivity″, in this example, does not pertain to discrimination of the two possible enantiomers which could be obtained (i.e., if the reaction had or had not changed the configuration). Chiral GC analysis revealed that the stereoselectivity was over 99%, the inversion product was obtained (compared to purchased reference substance).
(j) The Table heading ″Selectivity″, in this example, does not pertain to discrimination of the two possible enantiomers which could be obtained (i.e., if the reaction had or had not changed the configuration). Chiral GC analysis revealed that the stereoselectivity was over 98% in case of Example 80a and over 99% in case of Example 80b; in case of example 80c a racemic mixture was obtained. Seeberger reports a 6:1 mixture of diastereoisomers, which means a stereoselectivity of about 86%
(k) Examples 80d and 80e were done with 0.2M solution in DCM at 70° C. (instead of 0.5M in EtOAc at 50° C.). The Table heading ″Selectivity″, in this example, does not pertain to discrimination of the two possible enantiomers which could be obtained (i.e., if the reaction had or had not changed the configuration). Chiral GC analysis revealed that the stereoselectivity was over 97% in case of Example 80d and over 99% in case of Example 80e. Seeberger reports a 6:1 mixture of diastereoisomers, which means a stereoselectivity of about 86%.
(l) Examples 80f, 80g and 80h were done with 0.5M solution in DCM at 50° C. (instead of EtOAc). The stated selectivity does not discriminate between the two possible enantiomers which could be obtained if the conversion had or had not changed the configuration. Chiral GC analysis revealed that the stereoselectivity was over 97% in case of Example 80f, over 99% in example 80g and 50% (49:51) in example 80h. Seeberger reports a 6:1 mixture of diastereoisomers, which means a stereioselectivity of about 86%.
(m) Example 60A was done with Et₃N, Example 60B with DIPEA as base.

### Examples 100 to 108—Substrate Various Aldehydes

The examples shown in Table 4 were done according to (B) Fluorination Procedure with the aldehydes in the first column as substrate instead of phenylethanol and with following deviations from (B) Fluorination Procedure:
1 M aldehyde (instead of 0.5 M of phenylethanol in (B))
75° C. were used (instead of 50° C. in (B))
Used flow rates: \dot{V}ₗ=0.5 mL/min, \dot{V}_{g}=12.1 mLₙ/min (2 equiv SF₄), t_{Res} 552 sec (instead of \dot{V}₁=1 mL/min, \dot{V}_{g}=12.1 mLₙ/min (1 equiv SF₄)) t_{Res} 276 sec in (B))

The aldehydes reacted with SF₄ as shown in Scheme B, the oxo residue of the aldehyde group of the aldehyde was exchanged against two geminal fluorine atoms providing the respective difluoro compound.

TABLE 4

| | | conv | yield | isolated | sel |
|---|---|---|---|---|---|
| | | GC | GC | yield | GC |
| Ex | Substrate | [%] | [%] | [%] | [%] |
| 100 | 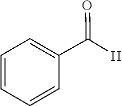 | 79 | 78 | na | 74 |
| 101 (b) | 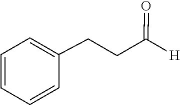 | 98 | 97 | 78 | 94 |
| 102 | 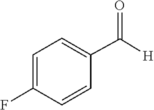 | 85 | 84 | na | 84 |
| 103 | 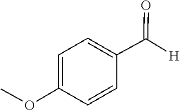 | >99 | 99 | 98 | 99 |
| 104A | 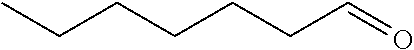 | 91 | 86 | na | 87 |
| 104B (a) | 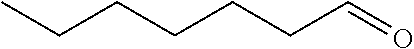 | 88 | 82 | na | 75 |
| 105 | 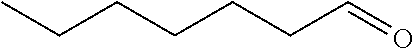 | 82 | 81 | na | 80 |
| 106 | 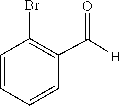 | 68 | 68 | na | 67 |
| 107 | 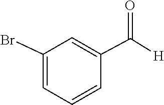 | 87 | 72 | na | 73 |
| 108 | 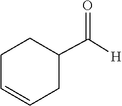 | 85 | 75 | na | 71 |

(a) Example 104B was done similar to Hasek with 1.5 equiv SF4 with a tres of 552 sec resulting in a reaction time of about 10 min and at 60° C.; Hasek in Table 5 reports a yield of 43% with 1.5 equiv of SF₄ a reaction temperature of 60° C. and a reaction time of 8 h
(b) Example 101 and its sampling were done without a sample loop, but pumping the substrate solution directly to the T-piece for 60 minutes, the stated conv GC, yield GC and sel GC were obtained right from the beginning and remained constant over the 60 min.

### Comparative Examples 1 to 14—Batch Procedure for the Fluorination According to Scheme 3

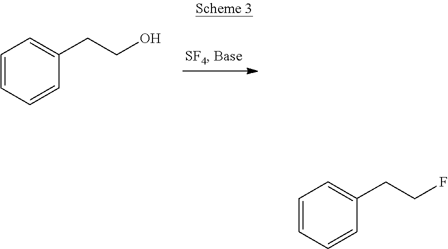 [SMILES: OCCc1ccccc1 ; FCCc1ccccc1]

### Comparative Example 1

Phenylethanol (549 mg, 4.5 mmol), pyridine as base (365 mg, 1 equiv), trifluorotoluene (131 mg, 20 mol %) and cyclohexane as solvent (4.5 mL, resulting in a concentration of 1 M of phenylethanol) were placed into a microwave vial (10 to 20 mL, Biotage, Sweden). The vial was sealed, cooled to −60° C., and flushed with Argon for 10 min. Then, the headspace in the vial was connected via a needle to a quench bottle of aqueous NaOH (10 wt %). Through a second needle SF₄ was condensed into the vial for a few minutes. After that, both needles were removed, the amount of SF₄ was determined using a balance, and the vial was allowed to warm up to room temperature while vigorous stirring. After 40 minutes stirring at room temperature, the headspace of the vial was connected through a needle to a quench solution, an aq. solution of NaOH (10 wt %), and the pressure was released. An aq. sat. solution of NaHCO₃ was added slowly to quench the reaction and the headspace was flushed with Argon for 5 min. Then, a sample from the organic phase was taken for HPLC analysis.

Comparative Examples 1 to 14 were done according to Comparative Example 1 with the difference as shown in Table 5.

TABLE 5

| | | | | conv | yield | sel |
|---|---|---|---|---|---|---|
| | | | SF₄ | HPLC | HPLC | HPLC |
| CompEx | Solvent | Base | equiv | [%] | [%] | [%] |
| 1 | Cyclohexane | Pyridine | 0.7 | 88 | 14 | 10 |
| 2 | Dichloromethane | Pyridine | 2.0 | 88 | 20 | 20 |
| 3 | Tetrahydrofuran | Pyridine | 3.4 | 40 | 10 | 7 |
| 4 | Diethylether | Pyridine | 3.1 | 80 | 14 | 11 |
| 5 | Cyclohexane | Et₃N | 2.7 | 97 | 52 | 56 |
| 6 | Cyclohexane | DIPEA | 1.8 | 98 | 27 | 28 |
| 7 | Cyclohexane | Piperidine | 1.3 | 87 | 42 | 47 |
| 8 | Cyclohexane | DBU | 1.2 | 96 | 29 | 30 |
| 9 | Dichloromethane | Et₃N | 2.9 | 98 | 33 | 33 |
| 10 | Cyclohexane | Et₃N | 2.4 | 84 | 34 | 36 |
| 11 | Cyclohexane | DIPEA | 1.8 | 96 | 39 | 42 |
| 12 (1) | EtOAc | Et₃N | 2.2 | 98 | 32 | 40 |
| 13 (2) | Cyclohexane | Pyridine | 4.4 | 85 | 19 | 20 |
| 14 (3) | Cyclohexane | Pyridine | 2 | >99 | 21 | 27 |

(1) Comparative Example 12 was done with these parameters:
   0.5 M substrate phenylethanol
   2.2 equiv SF₄ or more
   1 equiv Et₃N
   solvent EtOAc
   25° C. (same temperature as in Example 12)
   reaction time 60 min
Yield and selectivity are considerably lower than in Example 12.
Janzen reports a yield of 35% at a reaction temperature of −50° C.
(2) Comparative Example 13 was done with 6 M solution of phenylethanol; analytics were done with GC, not with HPLC; Janzen reports a yield of 35% at a reaction temperature of −50° C.
(3) Comparative Example 14 was done with 0.5 M solution of phenylethanol; analytics were done with GC, not with HPLC; Janzen reports a yield of 35% at a reaction temperature of −50° C.

### Examples 200 to 208—Substrate Cyclohexanone

Cyclohexanone was reacted as shown in Scheme 8 with SF₄ according to (B) Fluorination Procedure with cyclohexanone as substrate instead of phenylethanol; deviations from the protocol of the (B) Fluorination Procedure are given in Table 8.

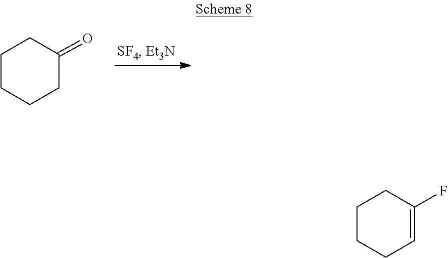 [SMILES: O=C1CCCCC1 ; FC1=CCCCC1]

TABLE 8

| | | | | Flow | Flow | | | | |
|---|---|---|---|---|---|---|---|---|---|
| | | | | Rate | Rate | | conv | yield | sel |
| | SF₄ | Et₃N | T | \dot{V}ₗ | \dot{V}_{g} | t_{Res} | GC | GC | GC |
| Ex | [equiv] | [equiv] | [° C.] | [mL/min] | [mLₙ/min] | [sec] | [%] | [%] | [%] |
| 200 | 1 | 1 | 75 | 0.5 | 6.1 | 552 | >99 | 86 | 81 |
| 201 | 2 | 1 | 75 | 0.5 | 12.1 | 552 | >99 | 87 | 80 |
| 202 | 2 | 2 | 75 | 0.5 | 12.1 | 552 | >99 | 85 | 80 |
| 203 | 3 | 3 | 75 | 0.5 | 18.2 | 552 | >99 | 81 | 74 |
| 204 | 1 | 1 | 30 | 0.5 | 6.1 | 552 | >99 | 84 | 77 |
| 205 | 2 | 2 | 30 | 0.5 | 12.1 | 552 | 99 | 83 | 76 |
| 206 | 1 | 1 | 50 | 0.5 | 6.1 | 552 | 99 | 86 | 79 |
| 207 | 2 | 2 | 50 | 0.5 | 12.1 | 552 | >99 | 84 | 77 |
| 208 (i) | 1 | 1 | 90 | 0.39 | 4.8 | 2385 | 99 | na | 81 |

(i) Example 208 was done with a reactor coil having 0.8 mm i.d., 15.5 mL volume)

### Examples 300 to 313—Substrate Phenylpropionic Acid and Benzoic Acid

Phenylpropionic acid was reacted as shown in Scheme 9 with SF₄ according to (B) Fluorination Procedure with phenylpropionic acid as substrate instead of phenylethanol; deviations from the protocol of the (B) Fluorination Procedure are given in Table 9.

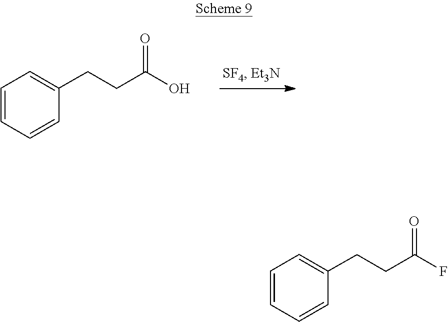 [SMILES: O=C(O)CCc1ccccc1 ; O=C(F)CCc1ccccc1]

TABLE 9

| | | | | Flow | Flow | | | | |
|---|---|---|---|---|---|---|---|---|---|
| | | | | Rate | Rate | | conv | yield | sel |
| | SF₄ | Et₃N | T | \dot{V}ₗ | \dot{V}_{g} | t_{Res} | GC | GC | GC |
| Ex | [equiv] | [equiv] | [° C.] | [mL/min] | [mLₙ/min] | [sec] | [%] | [%] | [%] |
| 300 | 2 | 2 | 75 | 0.5 | 12.1 | 552 | 99 | 95 | 94 |
| 301 | 3 | 3 | 75 | 0.5 | 18.2 | 552 | 98 | 91 | 86 |
| 302 | 4 | 4 | 75 | 0.5 | 24.2 | 552 | 99 | 91 | 91 |
| 303 (i) | 1 | 1 | 50 | 1 | 12.1 | 276 | 99 | 97 | 97 |

(i) isolated yield of Example 303 was 92%.

Benzoic acid was reacted as shown in Scheme 10 with SF₄ according to (B) Fluorination Procedure with Benzoic acid as substrate instead of phenylethanol; deviations from the protocol of the (B) Fluorination Procedure are given in Table 10.

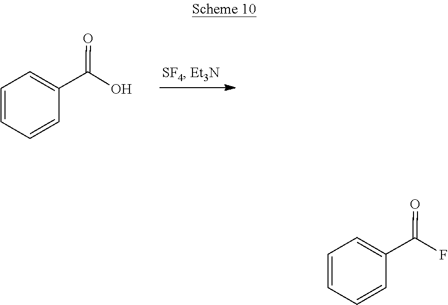 [SMILES: O=C(O)c1ccccc1 ; O=C(F)c1ccccc1]

TABLE 10

| | | | Flow | Flow | | | |
|---|---|---|---|---|---|---|---|
| | | | Rate | Rate | | | |
| | | | \dot{V}ₗ | \dot{V}_{g} | | conv | sel |
| | SF₄ | T | [mL/ | [mLₙ/ | t_{Res} | HPLC | HPLC |
| Ex | [equiv] | [° C.] | min] | min] | [sec] | [%] | [%] |
| 310 | 1 | 30 | 0.5 | 6.0 | 552 | 99 | >99 |
| 311 | 2 | 30 | 0.5 | 12.1 | 552 | >99 | >99 |
| 312 | 1 | 50 | 1 | 12.1 | 276 | >99 | >99 |
| 313 | 1 | 75 | 1 | 12.1 | 276 | >99 | >99 |

### Examples 330 to 334—Substrate: Various Acids

The examples shown in Table 13 were done according to (B) Fluorination Procedure with the substrates given in Table 13 instead of Phenylethanol.

The carboxylic acids reacted with SF₄ as shown in Scheme C, the hydroxyl residue of the carboxylic acid was exchanged against fluorine providing the respective acid fluoride.

TABLE 13

| | | conv | yield | isolated | sel |
|---|---|---|---|---|---|
| | | HPLC | HPLC | yield | HPLC |
| Ex | Substrate | [%] | [%] | [%] | [%] |
| 330 | 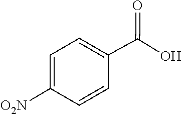 [SMILES: O=C(O)c1ccc([N+](=O)[O-])cc1] | 90 | na | na | 85 |
| 331 | 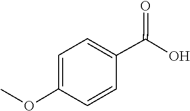 | >99 | na | na | 99 |
| 332 | 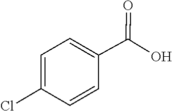 | >99 | na | na | 99 |
| 333 (a) | 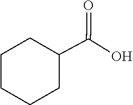 | >99 | 99 | na | >99 |
| 334 | 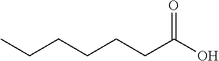 | >99 | 99 | na | >99 |

(a) Analyzed by GC for conversion, yield and selectivity.

### Examples 360—Substrate: Ketone

A substituted cyclohexanone was reacted as shown in Scheme D with SF₄ according to (B) Fluorination Procedure with the ketone shown in Table 14 as substrate instead of phenylethanol, the oxo residue of the keto group was exchanged against fluorine providing the respective vinyl fluoride.

TABLE 14

| | | conv | yield | sel |
|---|---|---|---|---|
| | | GC | GC | GC |
| Ex | Substrate | [%] | [%] | [%] |
| 360 | 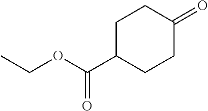 | >97 | 80 | 80 |

1. A method for deoxyfluorination of anyone of substrates a), b), c) and d), by a reaction of said substrate a), b), c) or d) with the fluorination reagent SF₄ in the presence of at least 1 equiv of a base, the equiv are molar equiv based on the molar amount of substrate;
the reaction is done in a continuous flow reaction mode;
wherein
substrate a) is a primary or secondary alcohol, or
substrate a) is a di-, tri- or tetra-peptide of natural alpha amino acids containing a primary or secondary aliphatic OH residue in its side chain,
substrate b) is an aldehyde,
substrate c) is a carboxylic acid, and
substrate d) is an unsubstituted or substituted cyclohexanone of formula (D6) or an unsubstituted or substituted cyclohexenone of formula (D6), or an unsubstituted or substituted cyclopentanone of formula (D5) or an unsubstituted or substituted cyclopentenone of formula (D5),
and wherein
in case of substrate a) the hydroxyl residue of the primary or secondary alcohol is exchanged against fluorine providing the respective (mono)fluorine compound;
in case of substrate b) the oxo residue of the aldehyde group of the aldehyde is exchanged against two geminal fluorine atoms providing the respective difluoro compound;
in case of substrate c) the hydroxyl residue of the carboxylic acid is exchanged against fluorine providing the respective acid fluoride;
in case of substrate d) the oxo residue of the keto group is exchanged against fluorine providing a vinyl fluoride compound of formula (D6-F) or of formula (D5-F) respectively according to Scheme D, 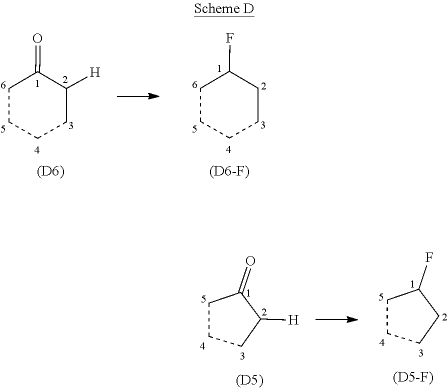
wherein in formula (D6) and formula (D6-F) the three endocyclic bonds C³-C⁴, C⁴-C⁵ and C⁵-C⁶ are either single bonds or one of said three bonds is a double bond and the other two bonds of said three bonds are single bonds, and
wherein in formula (D5) and formula (D5-F) the two endocyclic bonds C³-C⁴ and C⁴-C⁵ are either single bonds or one of said two bonds is a single bond and the other bond of said two bonds is a double bond.

2. The method for deoxyfluorination according to claim 1, wherein
the reaction temperature is from −25 to 200° C., preferably from −10 to 200° C., more preferably from 0 to 200° C., even more preferably from 10 to 200° C., especially from 10 to 175° C., more especially from 15 to 175° C., even more especially from 20 to 175° C.

3. The method for deoxyfluorination according to claim 1, wherein the reaction is done in a solvent.

4. The method for deoxyfluorination according to claim 3, wherein
the solvent is selected from the group consisting of benzene, chlorobenzene toluene, methylene chloride, dichloromethane, fluorotrichloromethane, tetrahydrofuran, C₅₋₁₀ alkane, C₁₋₄ carboxylic acid C₁₋₄ alkyl ester, and C₂₋₄ alkyl C₂₋₄ alkyl ether, preferably the solvent is selected from the group consisting of cyclohexane, C₂₋₄ carboxylic acid C₁₋₄ alkyl ester, and C₂₋₄ alkyl C₂₋₄ alkyl ether, in a particular embodiment, the solvent is selected from the group consisting of cyclohexane, ethyl acetate, butyl acetate, diisopropyl ether, methyl tert-butyl ether and isopropyl acetate, in a preferred particular embodiment the solvent is ethyl acetate or butyl acetate, in a more preferred particular embodiment the solvent is ethyl acetate.

5. The method for deoxyfluorination according to claim 3, wherein the solvent is not HF.

6. The method for deoxyfluorination according to claim 1, wherein the reaction is done without any addition of HF to the reaction.

7. The method for deoxyfluorination according to claim 1, wherein
the base is selected from the group consisting of pyridine, R10(R11)(R12)N, piperidine, pyridine and 1,8-Diazabicyclo[5.4.0]undec-7-ene (DBU), wherein
R10, R11 and R12 are identical or different and independently from each other selected from the group consisting of H and C₁₋₄ alkyl, preferably R10, R11 and R12 are identical or different and independently from each other selected from the group consisting of H, methyl, ethyl, n-propyl, and isopropyl, more preferably R10, R11 and R12 are identical or different and independently from each other ethyl or isopropyl, in a particular embodiment, the base is triethylamine (Et₃N) or N,N-diisopropylethylamine (DIPEA).

8. The method for deoxyfluorination according to claim 1, wherein
the substrate is mixed with the base prior to reaction with the SF₄.

9. The method for deoxyfluorination according to claim 3, wherein
for the reaction to take place a mixture of the substrate, the base and the solvent is mixed with SF₄.

10. The method for deoxyfluorination according to claim 3, wherein
the reaction is done at a pressure, also called reaction pressure, which is above the vapor pressure at the chosen reaction temperature of a mixture containing all components of the reaction mixture except for the SF₄, and/or
the reaction pressure is above the vapor pressure of SF₄ at the chosen reaction temperature;
preferably, the reaction pressure is above the vapor pressure of the reaction mixture at the chosen reaction temperature.

11. The method for deoxyfluorination according to claim 1, wherein
the substrate and the SF₄ are mixed continuously in a mixing device (MIXDEV) in the presence of the base;
MIXDEV is a T-piece, a microreactor, a mixing device, such as a static mixing device, or any combination thereof.

12. The method for deoxyfluorination according to claim 1, wherein
the reaction mixture is passed through a residence device (RESDEV) after having passed through MIXDEV.

13. The method for deoxyfluorination according to claim 1, wherein
that is the inner diameter of any channel in MIXDEV and/or RESDEV, through which the reaction mixture passes, is 2 mm or less, preferably 1 mm or less.

14. The method for deoxyfluorination according to claim 1, wherein
the reaction time is from 0.1 to 60 min, preferable from 0.2 to 50 min, more preferably from 0.4 to 40 min.

15. The method for deoxyfluorination according to claim 1, wherein
substrate a) is a primary or secondary alcohol of formula (A), wherein the hydroxyl residue of the primary or secondary alcohol is exchanged against fluorine providing the (mono)fluorine compound of formula (A-F) according to Scheme A; 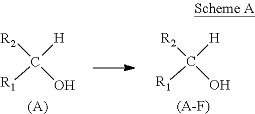
substrate b) is an aldehyde of formula (B), wherein the oxo residue of the aldehyde group of the aldehyde is exchanged against two geminal fluorine atoms providing the respective difluoro compound of formula (B-F) according to Scheme B; 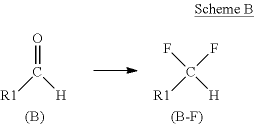
substrate c) is a carboxylic acid of formula (C), wherein the hydroxyl residue of the carboxylic acid is exchanged against fluorine providing the respective acid fluoride of formula (C-F) according to Scheme C 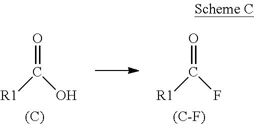
with regard to substrate d),
said unsubstituted or substituted cyclohexanone of formula (D6) or the unsubstituted or substituted cyclohexenone of formula (D6) is a compound of formula (D6-I),
said unsubstituted or substituted cyclopentanone of formula (D5) or an unsubstituted or substituted cyclopentenone of formula (D5) is a compound of formula (D5-I),
said vinyl fluoride compound of formula (D6-F) of Scheme D is a compound of formula (D6-F-I),
said vinyl fluoride compound of formula (D5-F) of Scheme D is a compound of formula (D5-F-I),
wherein the oxo residue of the keto group is exchanged against fluorine according to Scheme D-I, 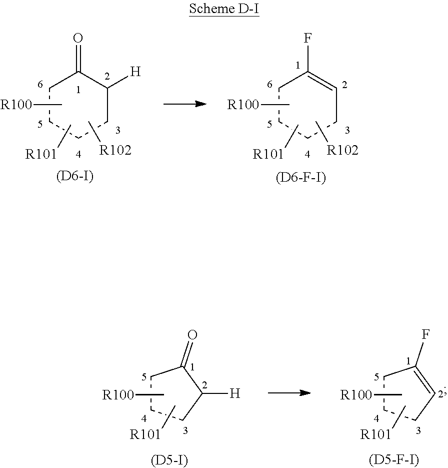
wherein in formula (D6-I) and formula (D6-F-I) the three endocyclic bonds C³-C⁴, C⁴-C⁵ and C⁵-C⁶ are either single bonds or one of said three bonds is a double bond and the other two bonds of said three bonds are single bonds, and
wherein in formula (D5-I) and formula (D5-F-I) the two endocyclic bonds C³-C⁴ and C⁴-C⁵ are either single bonds or one of said two bonds is a single bond and the other bond of said two bonds is a double bond,
or, alternatively,
substrate d) is a steroid of formula (GON) or a steroid of formula (CHOL) substituted with an oxo at C³ or at C¹⁷ of the steroid of formula (GON), or at C³ of the steroid of formula (CHOL), respectively; 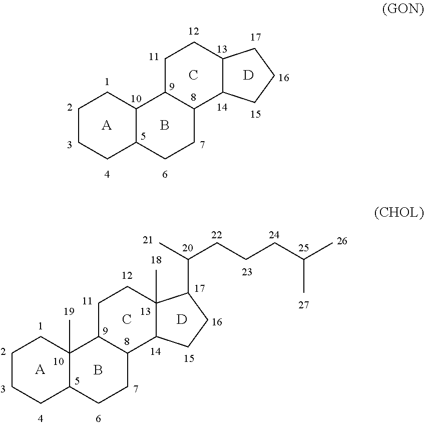
wherein
R1 and R2 are identical or different and independently from each other selected from the group consisting of
H, C₁₋₃₀ alkyl, C₂₋₃₀ alkenyl, F, Cl, Br, I, NO₂,
O—C₁₋₁₀ alkyl, O—C₂₋₁₀ alkenyl,
(O—CH₂—CH₂)₁₋₁₅₀—O—R6,
(O-propylene)₁₋₁₅₀-O—R6,
(O—CH₂—CH₂)₁₋₁₅₀—(O-propylene)₁₋₁₅₀(O—CH₂—CH₂)₀₋₁₅₀—O—R6,
(O-propylene)₁₋₁₅₀-(O—CH₂—CH₂)₁₋₁₅₀—(O-propylene)₀₋₁₅₀-O—R6;
C(O)H, C(O)—C₁₋₁₀ alkyl, C(O)—C₂₋₁₀ alkenyl,
COOH, C(O)—O—C₁₋₁₀ alkyl, C(O)—O—C₂₋₁₀ alkenyl,
C(O)—(O—CH₂—CH₂)₁₋₁₅₀—O—R6,
C(O)—(O-propylene)₁₋₁₅₀-O—R6,
C(O)—(O—CH₂—CH₂)₁₋₁₅₀—(O-propylene)₁₋₁₅₀(O—CH₂—CH₂)₀₋₁₅₀—O—R6,
C(O)—(O-propylene)₁₋₁₅₀-(O—CH₂—CH₂)₁₋₁₅₀—(O-propylene)₀₋₁₅₀-O—R6;
O—R5, S—R5, N(H)R5,
a ring RINGALIPH,
(Y1)-RINGALIPH,
aryl, and
(Y1)-aryl;
or
R1 and R2 together with the connecting C atom form a ring RINGALIPH;
R100, R101 and R102 are identical or different and independently from each other selected from the group consisting of
oxo, H, C₁₋₃₀ alkyl, C₂₋₃₀ alkenyl,
OH, SH, F, Cl, Br, I, NO₂,
O—C₁₋₁₀ alkyl, O—C₂₋₁₀ alkenyl,
(O—CH₂—CH₂)₁₋₁₅₀—O—R6,
(O-propylene)₁₋₁₅₀-O—R6,
(O—CH₂—CH₂)₁₋₁₅₀—(O-propylene)₁₋₁₅₀(O—CH₂—CH₂)₀₋₁₅₀—O—R6,
(O-propylene)₁₋₁₅₀-(O—CH₂—CH₂)₁₋₁₅₀—(O-propylene)₀₋₁₅₀-O—R6,
C(O)H, C(O)—C₁₋₁₀ alkyl, C(O)—C₂₋₁₀ alkenyl,
COOH, C(O)—O—C₁₋₁₀ alkyl, C(O)—O—C₂₋₁₀ alkenyl,
C(O)—(O—CH₂—CH₂)₁₋₁₅₀—O—R6,
C(O)—(O-propylene)₁₋₁₅₀-O—R6,
C(O)—(O—CH₂—CH₂)₁₋₁₅₀—(O-propylene)₁₋₁₅₀(O—CH₂—CH₂)₀₋₁₅₀—O—R6,
C(O)—(O-propylene)₁₋₁₅₀-(O—CH₂—CH₂)₁₋₁₅₀—(O-propylene)₀₋₁₅₀-O—R6,
O—R5, S—R5, N(H)R5,
aryl, and
(Y1)-aryl;
and wherein
any mentioned RINGALIPH is, independently from any other RINGALIPH,
a 4, 5 or 6 membered saturated or unsaturated, non-aromatic carbocyclic or heterocyclic residue, the heterocyclic residue containing 1 or 2 identical or different endocyclic heteroatoms Y2,
a steroid residue, or
a bicyclic spiro residue,
each ring of the bicyclic spiro residue is independently from the other ring a 4, 5 or 6 membered ring,
in case of a 4 membered ring of the bicyclic spiro residue the ring contains 0 or 1 endocyclic heteroatom Y2,
in case of a 5 or 6 membered ring of the bicyclic spiro residue the ring contains 0, 1 or 2 identical or different endocyclic heteroatoms Y2;
and
RINGALIPH is unsubstituted or substituted by one or more substituents independently from each other selected from the group consisting of
oxo, C₁₋₃₀ alkyl, C₂₋₃₀ alkenyl, OH, SH, F, Cl, Br, I, NO₂,
O—C₁₋₁₀ alkyl, O—C₂₋₁₀ alkenyl,
(O—CH₂—CH₂)₁₋₁₅₀—O—R6,
(O-propylene)₁₋₁₅₀-O—R6,
(O—CH₂—CH₂)₁₋₁₅₀—(O-propylene)₁₋₁₅₀(O—CH₂—CH₂)₀₋₁₅₀—O—R6,
(O-propylene)₁₋₁₅₀-(O—CH₂—CH₂)₁₋₁₅₀—(O-propylene)₀₋₁₅₀-O—R6,
C(O)H, C(O)—C₁₋₁₀ alkyl, C(O)—C₂₋₁₀ alkenyl,
COOH, C(O)—O—C₁₋₁₀ alkyl, C(O)—O—C₂₋₁₀ alkenyl,
C(O)—(O—CH₂—CH₂)₁₋₁₅₀—O—R6,
C(O)—(O-propylene)₁₋₁₅₀-O—R6,
C(O)—(O—CH₂—CH₂)₁₋₁₅₀—(O-propylene)₁₋₁₅₀(O—CH₂—CH₂)₀₋₁₅₀—O—R6,
C(O)—(O-propylene)₁₋₁₅₀-(O—CH₂—CH₂)₁₋₁₅₀—(O-propylene)₀₋₁₅₀-O—R6,
O—R5, S—R5, N(H)R5,
aryl, and
(Y1)-aryl;
any mentioned aryl is, independently from any other aryl,
a 5 or 6 membered monocyclic aryl residue,
a bicyclic aryl residue formed by a 5 or 6 membered ring fused with another 5 or 6 membered ring, or
an aryl residue wherein a 5 or 6 membered aromatic ring is fused with a 5 or 6 membered non-aromatic ring, said aryl residue being connected with either said aromatic ring or with said non-aromatic ring to the C atom of formula (A),
any 5 membered ring in the aryl contains 0, 1 or 2 identical or different endocyclic heteroatoms Y2,
any 6 membered ring in the aryl contains 0, 1, 2 or 3 identical or different endocyclic heteroatoms Y2;
and
aryl is unsubstituted or substituted by one or more identical or different substituents selected from the group consisting of
C₁₋₁₀ alkyl, C₂₋₁₀ alkenyl, F, Cl, Br, I, NO₂,
O—C₁₋₁₀ alkyl, O—C₂₋₁₀ alkenyl,
(O—CH₂—CH₂)₁₋₁₅₀—O—R6,
(O-propylene)₁₋₁₅₀-O—R6,
(O—CH₂—CH₂)₁₋₁₅₀—(O-propylene)₁₋₁₅₀(O—CH₂—CH₂)₀₋₁₅₀—O—R6,
(O-propylene)₁₋₁₅₀-(O—CH₂—CH₂)₁₋₁₅₀—(O-propylene)₀₋₁₅₀-O—R6,
C(O)H, C(O)—C₁₋₁₀ alkyl, C(O)—C₂₋₁₀ alkenyl,
COOH, C(O)—O—C₁₋₁₀ alkyl, C(O)—O—C₂₋₁₀ alkenyl,
C(O)—(O—CH₂—CH₂)₁₋₁₅₀—O—R6,
C(O)—(O-propylene)₁₋₁₅₀-O—R6,
C(O)—(O—CH₂—CH₂)₁₋₁₅₀—(O-propylene)₁₋₁₅₀(O—CH₂—CH₂)₀₋₁₅₀—O—R6,
C(O)—(O-propylene)₁₋₁₅₀-(O—CH₂—CH₂)₁₋₁₅₀—(O-propylene)₀₋₁₅₀-O—R6;
O—R5, S—R5, N(H)R5,
a terminal aryl, and
(Y1)-aryl which is a terminal aryl;
any mentioned heteroatom Y2 is, independently from any other Y2, selected from the group consisting of O, S, and saturated or unsaturated N, said saturated N is unsubstituted or substituted by R5;
any mentioned Y1 is, independently from any other Y1, a connecting group selected from the group consisting of
O, C₁₋₆ alkylene, C(O),
C(O)—C₁₋₆ alkylene-C(O),
(O—CH₂—CH₂)₁₋₁₅₀—O,
(O-propylene)₁₋₁₅₀-O,
(O—CH₂—CH₂)₁₋₁₅₀—(O-propylene)₁₋₁₅₀(O—CH₂—CH₂)₀₋₁₅₀—O,
(O-propylene)₁₋₁₅₀-(O—CH₂—CH₂)₁₋₁₅₀—(O-propylene)₀₋₁₅₀-O;
(O—CH₂—CH₂)₁₋₁₅₀—O—C(O)—C₁₋₆ alkylene-C(O),
(O—CH₂—CH₂)₁₋₁₅₀—O—C(O)—C₁₋₆ alkylene-C(O)—O,
(O—CH₂—CH₂)₁₋₁₅₀—(O-propylene)₁₋₁₅₀(O—CH₂—CH₂)₀₋₁₅₀—O—C(O)—C₁₋₆ alkylene-C(O),
(O-propylene)₁₋₁₅₀-O—C(O)—C₁₋₆ alkylene-C(O),
(O-propylene)₁₋₁₅₀-O—C(O)—C₁₋₆ alkylene-C(O)—O,
(O-propylene)₁₋₁₅₀-(O—CH₂—CH₂)₁₋₁₅₀—(O-propylene)₀₋₁₅₀-O—C(O)—C₁₋₆ alkylene-C(O),
C(O)—C₁₋₆ alkylene-C(O)—(O—CH₂—CH₂)₁₋₁₅₀—O,
C(O)—C₁₋₆ alkylene-C(O)—(O-propylene)₁₋₁₅₀-O,
C(O)—C₁₋₆ alkylene-C(O)—(O—CH₂—CH₂)₁₋₁₅₀—(O-propylene)₁₋₁₅₀(O—CH₂—CH₂)₀₋₁₅₀—O,
C(O)—C₁₋₆ alkylene-C(O)—(O-propylene)₁₋₁₅₀-(O—CH₂—CH₂)₁₋₁₅₀—(O-propylene)₀₋₁₅₀-O,
C(O)—O—C₁₋₆ alkylene-O—C(O), O—C(O)—C₁₋₆ alkylene-C(O)—O,
O—C(O)—C₁₋₆ alkylene-C(O)—(O—CH₂—CH₂)₁₋₁₅₀—O, and
O—C(O)—C₁₋₆ alkylene-C(O)—(O-propylene)₁₋₁₅₀-O;
any mentioned C₁₋₃₀ alkyl and any mentioned C₂₋₃₀ alkenyl is, independently from any other C₁₋₃₀ alkyl and C₂₋₃₀ alkenyl respectively, unsubstituted or substituted by 1, 2, 3, 4, 5, or 6 identical of different substituents selected from the group consisting of
oxo, OH, SH, F, Cl, Br, I, NO₂,
O—C₁₋₁₀ alkyl, O—C₂₋₁₀ alkenyl,
(O—CH₂—CH₂)₁₋₁₅₀—O—R6,
(O-propylene)₁₋₁₅₀-O—R6,
(O—CH₂—CH₂)₁₋₁₅₀—(O-propylene)₁₋₁₅₀(O—CH₂—CH₂)₀₋₁₅₀—O—R6,
(O-propylene)₁₋₁₅₀-(O—CH₂—CH₂)₁₋₁₅₀—(O-propylene)₀₋₁₅₀-O—R6,
C(O)H, C(O)—C₁₋₁₀ alkyl, C(O)—C₂₋₁₀ alkenyl,
COOH, C(O)—O—C₁₋₁₀ alkyl, C(O)—O—C₂₋₁₀ alkenyl,
C(O)—(O—CH₂—CH₂)₁₋₁₅₀—O—R6,
C(O)—(O-propylene)₁₋₁₅₀-O—R6,
C(O)—(O—CH₂—CH₂)₁₋₁₅₀—(O-propylene)₁₋₁₅₀(O—CH₂—CH₂)₀₋₁₅₀—O—R6,
C(O)—(O-propylene)₁₋₁₅₀-(O—CH₂—CH₂)₁₋₁₅₀—(O-propylene)₀₋₁₅₀-O—R6,
O—R5, S—R5, N(H)R5,
aryl, and
(Y1)-aryl;
any mentioned C₁₋₁₀ alkyl and any mentioned C₂₋₁₀ alkenyl is, independently from any other C₁₋₁₀ alkyl and C₂₋₁₀ alkenyl, unsubstituted or substituted by 1, 2, 3, 4, 5, or 6 identical or different substituents selected from a group consisting of oxo, OH, SH, F, Cl, Br, I, NO₂,
O—C₁₋₉ alkyl, O—C₂₋₉ alkenyl,
(O—CH₂—CH₂)₁₋₁₅₀—O—R6,
(O-propylene)₁₋₁₅₀-O—R6,
(O—CH₂—CH₂)₁₋₁₅₀—(O-propylene)₁₋₁₅₀(O—CH₂—CH₂)₀₋₁₅₀—O—R6,
(O-propylene)₁₋₁₅₀-(O—CH₂—CH₂)₁₋₁₅₀—(O-propylene)₀₋₁₅₀-O—R6
C(O)H, C(O)—C₁₋₉ alkyl, C(O)—C₂₋₉ alkenyl,
COOH, C(O)—O—C₁₋₉ alkyl, C(O)—O—C₂₋₉ alkenyl,
C(O)—(O—CH₂—CH₂)₁₋₁₅₀—O—R6,
C(O)—(O-propylene)₁₋₁₅₀-O—R6,
C(O)—(O—CH₂—CH₂)₁₋₁₅₀—(O-propylene)₁₋₁₅₀(O—CH₂—CH₂)₀₋₁₅₀—O—R6,
C(O)—(O-propylene)₁₋₁₅₀-(O—CH₂—CH₂)₁₋₁₅₀—(O-propylene)₀₋₁₅₀-O—R6,
O—R5, S—R5, and N(H)R5;
any mentioned R5 is, independently from any other R5, a protecting group for protecting OH, SH or NH₂;
any mentioned R6 is, independently from any other R6, H or R5;
preferably, substrate d) is a unsubstituted or substituted cyclohexanone of formula (D6-I) or a unsubstituted or substituted cyclopentanone of formula (D5-I);
more preferably, substrate d) is a unsubstituted or substituted cyclohexanone of formula (D6-I);
preferably,
if OH is to be protected then R5 is Fmoc, Boc, benzoyl, Cbz, C(O)—C₁₋₁₀ alkyl, TBDMS (tert-Butyldimethylsilyl), triisopropylsilyl, PNB (p-Nitrobenzyl), ONB (o-Nitrobenzyl), Bn (Benzyl), Al (Allyl), or tBu (tert-Butyl),
more preferably Fmoc, Boc, benzoyl, Cbz, C(O)—C₁₋₆ alkyl, PNB (p-Nitrobenzyl), ONB (o-Nitrobenzyl), Bn (Benzyl), Al (Allyl), or tBu (tert-Butyl),
even more preferably Fmoc, Boc, benzoyl, Cbz, C(O)—C₁₃ alkyl, or Bn (Benzyl);
if NH₂ is to be protected then R5 is Fmoc, Boc, benzoyl, Cbz, C(O)—C₁₋₁₀ alkyl, Bn (Benzyl), or Alloc (Allyloxycarbonyl),
more preferably Fmoc, Boc, benzoyl, Cbz, C(O)—C₁₋₆ alkyl, Bn (Benzyl), or Alloc (Allyloxycarbonyl);
if SH is to be protected then R5 is Fmoc, Boc, benzoyl, Cbz, C₁₋₂ alkyl, C(O)—C₁₋₁₀ alkyl, Bn (Benzyl), Meb (p-Methylbenzyl), Acm (Acetamidomethyl), or Trt,
more preferably Fmoc, Boc, benzoyl, Cbz, C₁₋₂ alkyl, C(O)—C₁₋₆ alkyl, Bn (Benzyl), Meb (p-Methylbenzyl), or Acm (Acetamidomethyl);
in particular, if OH is to be protected then R5 is Boc, benzoyl, Cbz, Fmoc, C(O)—C₁₋₆ alkyl or Bn (Benzyl);
preferably, in case of substrate b), R1 and R2 are not O—R5, S—R5, or N(H)R5;
preferably, in case of substrate c), R1 and R2 are not O—R5, S—R5, or N(H)R5;
more preferably, in case of both substrates b) and c), R1 and R2 are not O—R5, S—R5, or N(H)R5.

16. The method for deoxyfluorination according to claim 15, wherein
R1 and R2 are identical or different and independently from each other selected from the group consisting of
H, C₁₋₂₂ alkyl, C₂₋₂₂ alkenyl, F, Cl, Br, I,
O—C₁₋₆ alkyl, O—C₂₋₆ alkenyl,
C(O)H, C(O)—C₁₋₆ alkyl, C(O)—C₂₋₆ alkenyl,
COOH, C(O)—O—C₁₋₆ alkyl, C(O)—O—C₂₋₆ alkenyl,
O—R5, S—R5, N(H)R5,
a ring RINGALIPH,
(Y1)-RINGALIPH,
aryl, and
(Y1)-aryl;
or
R1 and R2 together with the connecting C atom form a ring RINGALIPH;
R100, R101 and R102 are identical or different and independently from each other selected from the group consisting of
oxo, H, C₁₋₂₂ alkyl, C₂₋₂₂ alkenyl,
OH, F, Cl, Br, I, NO₂,
O—C₁₋₆ alkyl, O—C₂₋₆ alkenyl,
C(O)H, C(O)—C₁₋₆ alkyl, C(O)—C₂₋₆ alkenyl,
COOH, C(O)—O—C₁₋₆ alkyl, C(O)—O—C₂₋₆ alkenyl,
O—R5, N(H)R5,
aryl, and
(Y1)-aryl;
and wherein
any mentioned RINGALIPH is, independently from any other RINGALIPH,
a 4, 5 or 6 membered saturated or unsaturated, non-aromatic carbocycle or heterocyclic residue, the heterocyclic residue containing 1 or 2 identical or different endocyclic heteroatoms Y2, or
a bicyclic spiro residue,
each ring of the bicyclic spiro residue is independently from the other ring a 5 or 6 membered ring,
in case of a 5 or 6 membered ring of the bicyclic spiro residue the ring contains 0, 1 or 2 identical or different endocyclic heteroatoms Y2;
and
RINGALIPH is unsubstituted or substituted by one or more substituents independently from each other selected from the group consisting of
oxo, C₁₋₂₂ alkyl, C₂₋₂₂ alkenyl, OH, F, Cl, Br, I, NO₂,
O—C₁₋₆ alkyl, O—C₂₋₆ alkenyl,
C(O)H, C(O)—C₁₋₆ alkyl, C(O)—C₂₋₆ alkenyl,
COOH, C(O)—O—C₁₋₆ alkyl, C(O)—O—C₂₋₆ alkenyl,
O—R5, N(H)R5,
aryl, and
(Y1)-aryl;
any mentioned aryl is, independently from any other aryl,
a 5 or 6 membered monocyclic aryl residue,
a bicyclic aryl residue formed by a 5 or 6 membered ring fused with a 6 membered ring, or
an aryl residue wherein a 5 or 6 membered aromatic ring is fused with a 5 or 6 membered non-aromatic ring, said aryl residue being connected with either said aromatic ring or with said non-aromatic ring to the C atom of formula (A),
any 5 membered ring in the aryl contains 0, 1 or 2 identical or different endocyclic heteroatoms Y2,
any 6 membered ring in the aryl contains 0, 1, 2 or 3 identical or different endocyclic heteroatoms Y2;
and
aryl is unsubstituted or substituted by one or more identical or different substituents selected from the group consisting of
C₁₋₆ alkyl, C₂₋₆ alkenyl, F, Cl, Br, I, NO₂,
O—C₁₋₆ alkyl, O—C₂₋₆ alkenyl,
C(O)H, C(O)—C₁₋₆ alkyl, C(O)—C₂₋₆ alkenyl,
COOH, C(O)—O—C₁₋₆ alkyl, C(O)—O—C₂₋₆ alkenyl,
O—R5, N(H)R5,
a terminal aryl, and
(Y1)-aryl which is a terminal aryl;
any mentioned heteroatom Y2 is, independently from any other Y2, selected from the group consisting of 0, S, and saturated or unsaturated N, said saturated N is unsubstituted or substituted by R5;
any mentioned Y1 is, independently from any other Y1, a connecting group selected from the group consisting of
O, C₁₋₆ alkylene, C(O),
(O—CH₂—CH₂)₁₋₂₀—O,
(O-propylene)₁₋₂₀-O,
(O—CH₂—CH₂)₁₋₂₀—O—C(O)—C₁₋₆ alkylene-C(O)—O,
(O-propylene)₁₋₂₀-O—C(O)—C₁₋₆ alkylene-C(O)—O,
O—C(O)—C₁₋₆ alkylene-C(O)—(O—CH₂—CH₂)₁₋₂₀—O, and
O—C(O)—C₁₋₆ alkylene-C(O)—(O-propylene)₁₋₂₀-0;
any mentioned C₁₋₂₂ alkyl and any mentioned C₂₋₂₂ alkenyl is, independently from any other C₁₋₂₂ alkyl and C₂₋₂₂ alkenyl respectively, unsubstituted or substituted by 1, 2, 3, 4, 5, or 6 identical of different substituents selected from the group consisting of oxo, OH, F, Cl, Br, I, NO₂,
O—C₁₋₆ alkyl, O—C₂₋₆ alkenyl,
C(O)H, C(O)—C₁₋₆ alkyl, C(O)—C₂₋₆ alkenyl,
COOH, C(O)—O—C₁₋₆ alkyl, C(O)—O—C₂₋₆ alkenyl,
O—R5, S—R5, N(H)R5,
aryl, and
(Y1)-aryl;
any mentioned C₁₋₆ alkyl and any mentioned C₂₋₆ alkenyl is, independently from any other C₁₋₆ alkyl and C₂₋₆ alkenyl, unsubstituted or substituted by 1, 2, 3, 4, 5, or 6 identical or different substituents selected from a group consisting of
oxo, OH, F, Cl, Br, I, NO₂,
O—C₁₋₄ alkyl, O—C₂₋₃ alkenyl,
C(O)H, C(O)—C₁₋₄ alkyl, C(O)—C₂₋₃ alkenyl,
COOH, C(O)—O—C₁₋₄ alkyl, C(O)—O—C₂₋₃ alkenyl,
O—R5, S—R5, and N(H)R5.

17. The method for deoxyfluorination according to claim 1, wherein
the compounds of formulas (D6), (D6-F), (D5), and (D5-F) of claim 1 and the compounds of formulas (D6-I), (D6-F-I), (D5-I) and (D5-F-I) of claim 15 are in an alpha position to the keto group not substituted by an exocyclic C(O), said alpha position to the keto group is the C² and the C⁶ in formulas (D6), (D6-F) and in formulas (D6-I) and (D6-F-I), and the C² and the C⁵ in formulas (D5), (D5-F) and in formulas (D5-I) and (D5-I-F), respectively;
and/or
the C⁶ in formula (D6) (D6-F) and in formula (D6-I) and (D6-I-F) and the C⁵ in formula (D5), (D5-F) and in formula (D5-I) and (D5-I-F) is not substituted by oxo;
and/or
the compounds of formulas (D6), (D6-F), (D5), and (D5-F) and the compounds of formulas (D6-I), (D6-F-I), (D5-I) and (D5-F-I) are not substituted by oxo in beta position to the keto group, said beta position can be an endocyclic beta position or it can be an exocyclic beta position in case of respective substitution;
preferably,
the compounds of formulas (D6), (D6-F), (D5) and (D5-F) and the compounds of formulas (D6-I), (D6-F-I), (D5-I) and (D5-F-I) are not substituted by oxo in alpha and/or in beta position to the keto group, said beta position can be an endocyclic beta position or it can be an exocyclic beta position in case of respective substitution, and/or
R100, R101 and R102 are not oxo.

18. The method for deoxyfluorination according to claim 15, wherein
when any RINGALIPH is a cyclohexane, cyclopentane or cyclobutane and is substituted by an oxo residue, said oxo residue forms together with the C atom an endocyclic C(O) residue, then
RINGALIPH is not substituted by oxo in alpha position of said endocyclic C(O) residue, and/or
RINGALIPH is not substituted by oxo in the beta position of said endocyclic C(O) residue, said beta position can be an endocyclic beta position or it can be an exocyclic beta position in case of respective substitution;
preferably,
when any RINGALIPH is a cyclohexane, cyclopentane or cyclobutane then RINGALIPH is not substituted by oxo;
more preferably,
RINGALIPH is not substituted by oxo.

19. The method for deoxyfluorination according to claim 15, wherein
when in substrate a) any one of R1 and R2 is said aryl or is said C₂₋₃₀ alkenyl, wherein said C₂₋₃₀ alkenyl is substituted with aryl and said C₂₋₃₀ alkenyl together with its substituting aryl represents a vinylogous aryl,
then any Y2 in said aryl and in said vinylogous aryl is not an endocyclic unsaturated N and is not an endocyclic saturated unsubstituted N.

20. The method for deoxyfluorination according to claim 15, wherein
any Y2 in any aryl and in any terminal aryl is not an endocyclic unsaturated N and is not unsubstituted saturated N, and/or
any Y2 in any RINGALIPH does is not an endocyclic unsaturated N and is not unsubstituted saturated N, and/or
any Y2 is not unsaturated N and is not unsubstituted saturated N.
